(19) United States
(12) Patent Application Publication
(10) Pub. No.: US 2006/0058236 A1
(43) Pub. Date: Mar. 16, 2006

(54) Endogenously-formed conjugate of albumin

(76) Inventors: Maria U. Hutchins, Mountain View, CA (US); Radwan Kiwan, Albany, CA (US); Samuel Zalipsky, Redwood City, CA (US)

Correspondence Address: PERKINS COIE LLP, P.O. BOX 2168, MENLO PARK, CA, 94026, US

(21) Appl. No.: 11/217,536

(22) Filed: Aug. 31, 2005

Related U.S. Application Data
(60) Provisional application No. 60/607,110, filed on Sep. 3, 2004.

(51) Int. Cl. A61K 38/38

(52) U.S. Cl. 514/12

(57) ABSTRACT

A conjugate formed in vivo and comprised of endogenous albumin and an amine-containing compound, such as a protein or a drug, is described. The conjugate is formed by in vivo cleavage of a polymer-dithiobenzyl-therapeutic agent conjugate to form an albumin-dithiobenzyl-therapeutic agent conjugate. The dithiol moiety of the albumin-therapeutic agent conjugate is cleaved in vivo to yield the free therapeutic agent in native form.

### CROSS-REFERENCE TO RELATED APPLICATIONS

This application claims the benefit of U.S. Provisional Application No. 60/607,110, filed Sep. 3, 2004, incorporated herein by reference in its entirety.

### TECHNICAL FIELD

The subject matter described herein relates to an endogenously-formed conjugate comprised of a therapeutic agent and endogenous albumin, and to methods of providing a therapeutic agent in the form of a conjugate comprised of the therapeutic agent and endogenous albumin.

### BACKGROUND

Human serum albumin is a multifunctional protein found in the bloodstream. It is an important factor in the regulation of plasma volume and tissue fluid balance through its contribution to the colloid osmotic pressure of plasma. Albumin normally constitutes 50-60% of plasma proteins and because of its relatively low molecular weight (66,500 Daltons), exerts 80-85% of the colloidal osmotic pressure of the blood. Albumin regulates transvascular fluid flux and hence, intra and extravascular fluid volumes, and transports lipid and lipid-soluble substances. Albumin solutions are frequently used for plasma volume expansion and maintenance of cardiac output in the treatment of certain types of shock or impending shock including those resulting from burns, surgery, hemorrhage, or other trauma or conditions in which a circulatory volume deficit is present.

Albumin has a blood circulation half-life of approximately two weeks and is designed by nature to carry lipids and other molecules. A hydrophobic binding pocket and a free thiol cysteine residue (Cys34) are features that enable this function. Due to its low pKa (approx. 7) Cys34 is one of the more reactive thiol groups appearing in human plasma. The Cys34 of albumin also accounts for the major fraction of thiol concentration in blood plasma (over 80%) (Kratz et al., J. Med. Chem., 45(25):5523-33 (2002)). The ability of albumin through its reactive thiol to act as a carrier has been utilized for therapeutic purposes. For example, attachment of drugs to albumin to improve the pharmacological properties of the drugs has been described (Kremer et al., Anticancer Drugs, 13:(6):615-23 (2002); Kratz et al., J. Drug Target., 8(5):305-18 (2000); Kratz et al., J. Med. Chem., 45(25):5523-33 (2002); Tanaka et al., Bioconjug. Chem., 2(4):261-9 (1991); Dosio et al., J. Control. Release, 76(1-2):107-17 (2001); Dings et al., Cancer Lett., 194(1):55-66 (2003); Wunder et al., J. Immunol., 170(9):4793-801 (2003); Christie et al., Biochem. Pharmacol., 36(20):3379-85 (1987)). The attachment of peptide and protein therapeutics to albumin has also been described (Holmes et al., Bioconjug. Chem., 11 (4):439-44 (2000), Leger et al., Bioorg. Med. Chem. Lett., 13(20):3571-5 (2003); Paige et al., Pharm. Res., 12(12):1883-8 (1995)). Conjugates of albumin and interferon-alpha (Albuferon™) and of albumin and human growth hormone (Albutropin™) and of albumin and interleukin-2 (Albuleukin™) are being tested for therapeutic effectiveness. The art also describes the use of standard recombinant molecular biology techniques to generate an albumin-protein fusion (U.S. Pat. No. 6,548,653). All but the latter conjugates with albumin involve ex vivo conjugate formation with an exogenous albumin. Potential drawbacks to using exogenous sources of albumin are contamination or an immunogenic response.

In vivo attachment of therapeutic agents to albumin has also been described, where, for example, a selected peptide is modified prior to administration to allow albumin to bind to the peptide. This approach is described using dipeptidyl peptidase IV-resistant glucagon-like-peptide-1 (GLP-1) analogs (Kim et al., Diabetes, 52(3):751-9 (2003)). A specific linker ([2-[2-[2-maleimido-propionamido-(ethoxy)-ethoxy]-acetamide) was attached to an added carboxyl-terminal lysine on the peptide to enable a cysteine residue of albumin to bind with the peptide. Others have investigated attaching specific tags to peptides or proteins in order to increase their binding to albumin in vivo (Koehler et al., Bioorg Med. Chem. Lett., 12(20):2883-6 (2002); Dennis et al., J. Biol. Chem., 277(38):35035-35043 (2002)); Smith et al., Bioconjug. Chem., 12:750-756 (2001)). A similar approach has been used with small molecule drugs, where a derivative of the drug was designed specifically to have the ability to bind with a cysteine residue of albumin. For example, this pro-drug strategy has been used for doxorubicin derivatives where the doxorubicin derivative is bound to endogenous albumin at its cysteine residue at position 34 (Cys34; Kratz et al., J Med. Chem., 45(25): 5523-33 (2002)). The in vivo attachment of a therapeutic agent to albumin has the advantage, relative to the ex vivo approach described above, in that endogenous albumin is used, thus obviating problems associated with contamination or an immunogenic response to the exogenous albumin. Yet, the prior art approach of in vivo formation of drug conjugates with endogenous albumin involves a permanent covalent linkage between the drug and the albumin. To the extent the linkage is cleavable or reversible, the drug or peptide released from the conjugate is in a modified form of the original compound.

It would be desirable to provide a conjugate of a therapeutic agent with endogenous albumin where the conjugate is (i) formed in vivo and (ii) reversible in vivo to yield the therapeutic agent in its native form.

The foregoing examples of the related art and limitations related therewith are intended to be illustrative and not exclusive. Other limitations of the related art will become apparent to those of skill in the art upon a reading of the specification and a study of the drawings.

### SUMMARY

Accordingly, in one aspect, a method for delivering a therapeutic agent in the form of a conjugate with albumin is provided. The method comprises administering to a subject a compound of the form polymer-disulfide-therapeutic agent, wherein said therapeutic agent comprises at least one amine moiety. Administration of the compound achieves formation of a conjugate comprised of the subject's endogenous albumin and the therapeutic agent.

In one embodiment, the polymer-disulfide-therapeutic agent conjugate is a polymer-dithiobenzyl-therapeutic agent conjugate having the structure: 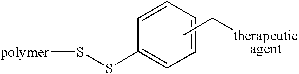
where orientation of CH₂-therapeutic agent is selected from the ortho position and the para position.

In another embodiment, the amine-containing therapeutic agent is selected from a protein and a drug. In preferred embodiments, the therapeutic agent is a protein having a drug or a protein having a molecular weight of less than about 45 kDa, more preferably of less than 30 kDa, and still more preferably of 15 kDa or less.

The polymer, in a preferred embodiment, is polyethylene glycol or a modified polyethyleneglycol.

In another aspect, a prodrug for treatment of a subject is described, the prodrug being comprised of the subject's endogenous albumin and a therapeutic agent comprising at least one amine moiety, the albumin and the therapeutic agent joined by a disulfide.

In yet another aspect, a method for extending the blood circulation lifetime of a therapeutic agent is contemplated, the method involving administering a polymer-disulfide-therapeutic agent conjugate as described above to achieve formation of a prodrug conjugate comprised of endogenous albumin and the therapeutic agent.

In addition to the exemplary aspects and embodiments described above, further aspects and embodiments will become apparent by reference to the drawings and by study of the following descriptions.

### BRIEF DESCRIPTION OF THE DRAWINGS

FIG. 1 shows a reaction scheme for in vivo formation of endogenous albumin and a therapeutic agent, where the therapeutic agent is administered to a subject in the form of a polymer-dithiobenzyl-therapeutic agent conjugate (polymer-DTB-therapeutic agent), and an albumin-DTB-therapeutic agent conjugate is formed in vivo, for eventual release of the therapeutic agent in its native form;

FIGS. 2A-2C show synthetic reaction schemes for preparation of a methoxy-polyethylene glycol (mPEG)-DTB-therapeutic agent conjugate (FIG. 2A), subsequent formation of an albumin-DTB-therapeutic agent conjugate (FIG. 2B), and decomposition of the albumin-DTB-therapeutic agent conjugate to release the native therapeutic agent (FIG. 2C);

FIGS. 3A-3B are HPLC traces for conjugates of polymer-DTB-lysozyme incubated in cysteine for various times between 10 minutes and 47 hours, where the conjugates were mPEG_{5K}-DTB-lysozyme (FIG. 3A) and mPEG_{12K}-DTB-lysozyme (FIG. 3B);

FIGS. 3C-3D are HPLC traces for conjugates of polymer-DTB-lysozyme incubated in BSA for various times between 10 minutes and 47 hours, where the conjugates were mPEG_{5K}-DTB-lysozyme (FIG. 3C) and mPEG_{12K}-DTB-lysozyme (FIG. 3D);

FIGS. 4A-4B are plots showing the percent of remaining conjugate as a function of time, in hours, upon incubation in cysteine (FIG. 4A) or in BSA (BSA) (FIG. 4B), for conjugates of mPEG_{12K}-DTB-lysozyme (triangles) and mPEGSK-DTB-lysozyme (diamonds);

FIGS. 4C-4D are plots showing the percent of regenerated lysozyme as a function of time, in hours, upon incubation in cysteine (FIG. 4C) or in BSA (FIG. 4C), for conjugates of mPEG_{12K}-DTB-lysozyme (triangles) and mPEG_{5K}-DTB-lysozyme (diamonds);

FIGS. 5A-5B are HPLC traces for the mPEG_{5K}-DTB-lysozyme conjugate incubated at room temperature in 4% BSA for 24 hours before (FIG. 5A) and after (FIG. 5B) passing the sample over a Q-spin column;

FIGS. 5C-5D are HPLC traces for the mPEG_{12K}-DTB-lysozyme conjugate incubated at room temperature in 4% BSA for 24 hours before (FIG. 5C) and after (FIG. 5D) passing the sample over a Q-spin column;

FIG. 6 shows an HPLC trace of sample resulting from incubation of mPEG_{5K}-DTB-lysozyme (1:1) conjugate with BSA for 2 days;

FIG. 7 is an SDS-PAGE gel of a sample resulting from incubation of mPEG_{5K}-DTB-lysozyme (1:1) conjugate with BSA for 2 days, where the fraction identifiers correspond to the peak identifiers indicated on the HPLC trace in FIG. 6;

FIG. 8 is an SDS-PAGE gel of a sample resulting from incubation of mPEG_{5K}-DTB-lysozyme (1:1) conjugate with BSA for 2 days and further incubated with mercaptoethanol, where the fraction identifiers correspond to the peak identifiers indicated on the HPLC trace in FIG. 6;

FIG. 9 shows a MALDI-TOF MS spectra of purified fraction E2 (identified in FIG. 6) corresponding to disulfide-linked albumin-lysozyme adduct of molecular weight 81 KDa.;

FIGS. 10A-10C show fluorescently labeled mPEG_{5K}-DTB-lysozyme conjugates incubated in the presence of rat plasma at 37° C. Samples were quenched according to the timecourse indicated and run on SDS-PAGE, non-reducing gels (FIG. 10A). FIG. 10B shows the same gel stained for total protein. FIG. 10C shows the quantitation of fluorescently-labeled species expressed relative to the total fluorescently-labeled species at each time point.

FIGS. 11A-11B show fluorescently labeled mPEG_{5K}-DTB-lysozyme conjugates incubated in the presence of bovine serum albumin (BSA) at 37° C. Samples were quenched according to the timecourse indicated and run on SDS-PAGE, non-reducing gels (FIG. 11A). FIG. 11B shows the quantitation of fluorescently-labeled species expressed relative to the total fluorescently-labeled species at each time point.

FIGS. 12A-12C show fluorescently labeled mPEG_{12K}-DTB-lysozyme conjugates incubated in the presence of rat plasma at 37° C. Samples were quenched according to the timecourse indicated and run on SDS-PAGE, non-reducing gels (FIG. 12A). FIG. 12B shows the same gel stained for total protein. FIG. 12C shows the quantitation of fluorescently-labeled species expressed relative to the total fluorescently-labeled species at each time point.

FIGS. 13A-13C show fluorescently labeled mPEG_{12K}-DTB-lysozyme conjugates incubated in the presence of bovine serum albumin (BSA) at 37° C. Samples were quenched according to the timecourse indicated and run on SDS-PAGE, non-reducing gels (FIG. 13A). FIG. 13B shows the same gel stained for total protein. FIG. 13C shows the quantitation of fluorescently-labeled species expressed relative to the total fluorescently-labeled species at each time point.

FIG. 14A is an SDS-PAGE gel of mPEG_{12K}-DTB-Epo+HSA (Lane 1); mPEG_{12K}-Epo+HSA (Lane 2); HSA+excess mPEG_{12K}-DTB-Glycine (Lane 3); HSA (Lane 4); mPEG_{12K}-DTB-Epo (Lane 5); mPEG_{12K}-DTB-Epo+2 mM Cysteine (Lane 6); Epo (Lane 7);

FIG. 14B is an immunoblot probed with anti-HSA where Lanes 1-7 correspond to the same samples in the SDS-PAGE gel of FIG. 14A;

FIGS. 15A-15C show data for fluorescently labeled mPEG_{12K}-DTB-Epo conjugates incubated in the presence of rat plasma at 37° C. Samples were quenched according to the timecourse indicated and run on SDS-PAGE, non-reducing gels (FIG. 15A). FIG. 15B shows the same gel stained for total protein. FIG. 15C shows the quantitation of fluorescently-labeled species expressed relative to the total fluorescently-labeled species at each time point;

FIGS. 16A-16C show data for fluorescently labeled mPEG_{30K}-DTB-Epo conjugates incubated in the presence of rat plasma at 37° C. Samples were quenched according to the timecourse indicated and run on SDS-PAGE, non-reducing gels (FIG. 16A). FIG. 16B shows the same gel stained for total protein. FIG. 16C shows the quantitation of fluorescently-labeled species expressed relative to the total fluorescently-labeled species at each time point;

FIGS. 17A-17C show data for fluorescently labeled mPEG_{30K}-DTB-Epo conjugates incubated in the presence of bovine serum albumin (BSA) at 37° C. Samples were quenched according to the timecourse indicated and run on SDS-PAGE, non-reducing gels (FIG. 17A). FIG. 17B shows the same gel stained for total protein. FIG. 17C shows the quantitation of fluorescently-labeled species expressed relative to the total fluorescently-labeled species at each time point;

FIGS. 18A-18C show data of a non-cleavable fluorescent mPEG_{30K}-lysine-NBD (7-nitrobenz-2-oxa-1,3-diazole) molecule incubated at 37° C. in the presence of bovine serum albumin at equimolar (Lanes 1-5) or 10-fold excess fluorophore (Lanes 6-10). Samples were quenched according to the timecourse indicated and run on SDS-PAGE, non-reducing gels (FIG. 18A). FIG. 18B is the same gel stained for PEG with iodine. FIG. 18C is the same gel then stained for protein;

FIGS. 19A-19F show data of a fluorescent mPEG_{30K}-DTB-lysine-NBD molecule incubated at 37° C. in the presence of bovine serum albumin at equimolar relative concentration. Samples were quenched according to the timecourse indicated and run on SDS-PAGE, non-reducing gels (FIGS. 19A, 19D). FIGS. 19B, 19E are the same gels stained for PEG with iodine. FIGS. 19C, 19F are the same gels then stained for protein;

FIGS. 20A-20D show data of a fluorescent mPEG_{30K}-DTB-lysine-NBD molecule incubated at 37° C. in the presence of bovine serum albumin at equimolar relative concentration. Samples were quenched according to the timecourse indicated and run on SDS-PAGE, non-reducing gels (FIG. 20A). FIG. 20B is the same gel stained for PEG with iodine. FIG. 20C is the same gel then stained for protein. FIG. 20D shows the quantitation of NBD species (from FIG. 20A gel) at each time point;

FIG. 21 shows the concentration of active lysozyme, in μg/mL, as a function of incubation time, in minutes, of the conjugate mPEG_{5K}-DTB-lysozyme with cysteine (squares), BSA (circles), or saline (triangles); and

FIG. 22 shows the pharmacokinetic profile obtained in rats intravenously dosed with I¹²⁵-lysozyme, I¹²⁵-labeled mPEG_{12K}-lysozyme, or I¹²⁵-labeled mPEG_{12K}-DTB-lysozyme.

### DETAILED DESCRIPTION

### I. Definitions and Abbreviations

“Protein” as used herein refers to a polymer of amino acids and does not refer to a specific length of a polymer of amino acids. Thus, for example, the terms peptide, polypeptide, oligopeptide, and enzyme are included within the definition of protein. This term also includes post-expression modifications of the protein, for example, glycosylations, acetylations, phosphorylations, and the like.

“Amine-containing” intends any compound having a moiety derived from ammonia by replacing one or two of the hydrogen atoms by alkyl or aryl groups to yield general structures RNH₂ (primary amines) and R₂NH (secondary amines), where R is any therapeutic moiety.

“Polymer” as used herein refers to a polymer having moieties soluble in water, which lend to the polymer some degree of water solubility at room temperature, i.e., the polymer is a hydrophilic polymer. Exemplary hydrophilic polymers include polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018. A preferred polymer is poly(ethyleneglycol) (PEG) and modified versions of PEG, such as methoxyPEG (mPEG). The molecular weight of the polymer is widely variable, and a typical range for mPEG is from 1,000 Daltons to 50,000 Daltons, more preferably, from 1,500 Daltons to 30,000 Daltons. In other embodiments, an mPEG molecular weight of less than about 30,000 Daltons is contemplated.

Reference to a polymer, drug, or therapeutic agent in the form of a “polymer-DTB-therapeutic agent conjugate” or to a “polymer-DTB-drug conjugate” or to an “albumin-therapeutic agent conjugate” or “albumin-drug conjugate” intends that the polymer, drug, or therapeutic agent is modified in some manner for conjugate formation, the modification including but not limited to addition of a functional group or loss of one or more chemical entities upon reaction with to form the conjugate.

Abbreviations: PEG, poly(ethylene glycol); mPEG, methoxy-PEG; DTB, dithiobenzyl; mDTB, methoxyDTB; EtDTB, ethoxyDTB; Epo, Erythropoietin; HSA, human serum albumin; BSA, bovine serum albumin; Cys, cysteine; SDS-PAGE, sodium dodecyl sulfate polyacrylamide gel electrophoresis; HPLC, high pressure liquid chromatography; MALDI-TOF MS, matrix assisted laser desorption/ionization time of flight mass spectrometry; kDa, kilodaltons; EDTA, ethylenediaminetetraacetic acid; NBD, (7-nitrobenz-2-oxa-1,3-diazole).

### II. Method of Conjugate Formation

In one aspect, a method for the in vivo formation of a compound comprised of endogenous albumin and a therapeutic agent is provided. The therapeutic agent can be any entity with an amine group, and exemplary entities are given below. It will be appreciated that conjugate formation between the two species, endogenous albumin and the therapeutic agent, results in modification of the endogenous albumin and/or the agent. Use of the terms “endogenous albumin” and “therapeutic agent” in the context of the conjugate intends residues of these species that comprise the conjugate. Formation of the in vivo adduct achieves an increased blood circulation lifetime of the therapeutic agent by virtue of its coupling with endogenous albumin. Thus, the method provides a solution to the problems associated with the short blood circulation time often observed with macromolecular biological therapeutics, and in particular, polypeptides, as well as low molecular weight drugs common in the pharmaceutical industry. By attaching endogenous albumin for use as a carrier protein, the lifetime of the polypeptide or drug can be extended, with the additional benefit of little, if any immunogenic response, since the patient's own albumin is used in formation of the conjugate.

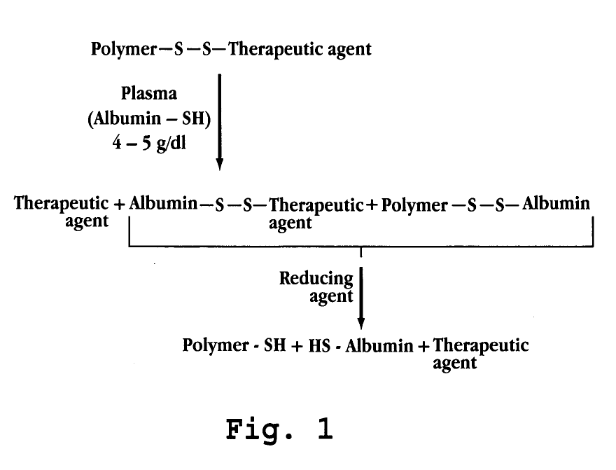

FIG. 1 generally outlines formation of an albumin-therapeutic agent adduct in vivo and using endogenous albumin. A polymer-disulfide-therapeutic agent conjugate is prepared and administered to a subject. Typically, the conjugate is administered intravenously, but any parenteral route is suitable. The polymer-disulfide-therapeutic agent conjugate is reduced in the blood stream due to the presence of small molecule thiols in the blood stream, such as glutathione, cysteine, homocysteine, cysteinyl-cysteine, and albumin. Reduction of the polymer-disulfide-therapeutic agent conjugate in the presence of albumin in the plasma results in formation of an albumin-disulfide-therapeutic agent adduct, along with formation of a polymer-disulfide-albumin adduct, and release of the therapeutic agent in free form. The cysteine residue at position 34 in albumin (Cys34) has a free thiol that is not involved in internal disulfide bonding, and which accounts for the majority of free thiol in the bloodstream. Approximately 60% of albumin molecules are believed to be in the free thiol form in plasma. The albumin-disulfide-therapeutic agent conjugate continues to circulate in the blood, and with time is reduced by the small molecule thiols in the blood. Reduction of the albumin-disulfide-therapeutic agent conjugate in the blood yields release of the therapeutic agent in its native form in the blood.

As noted above, the therapeutic agent can be virtually any amine-containing compound. The compound can be a therapeutic agent or a diagnostic agent or a compound with neither therapeutic nor diagnostic activity but desirous of in vivo administration. In preferred embodiments, the amine-containing therapeutic agent is a drug or a protein. A wide variety of therapeutic drugs have a reactive amine moiety, such as mitomycin C, bleomycin, doxorubicin and ciprofloxacin, and the method contemplates any of these drugs with no limitation. The molecular weight of such drugs is typically less than 2 kDa, often less than 1 kDa. Most proteins contain reactive amino groups, and proteins for therapeutic purposes or for targeting purposes are known in the art. Exemplary proteins can be naturally occurring or recombinantly produced polypeptides. Small, human recombinant polypeptides are preferred, and polypeptides in the range of 0.1-45 kDa, more preferably 0.5-30 kDa, still more preferably of 1-15 kDa are preferred. Molecular weights of polypeptides are reported in the literature or can be determine experimentally using routine methods.

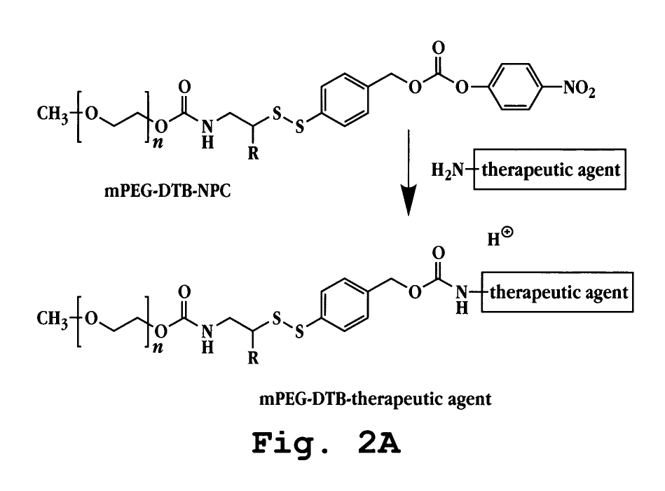

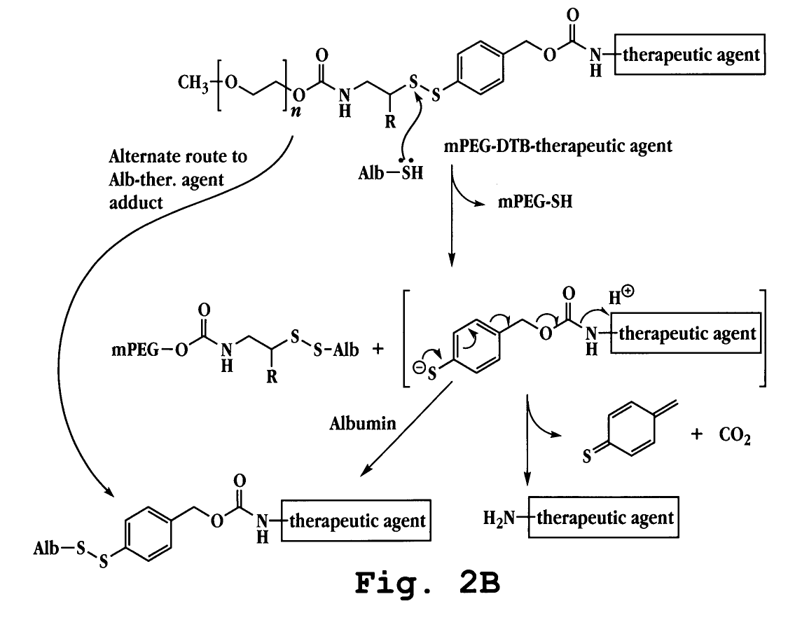

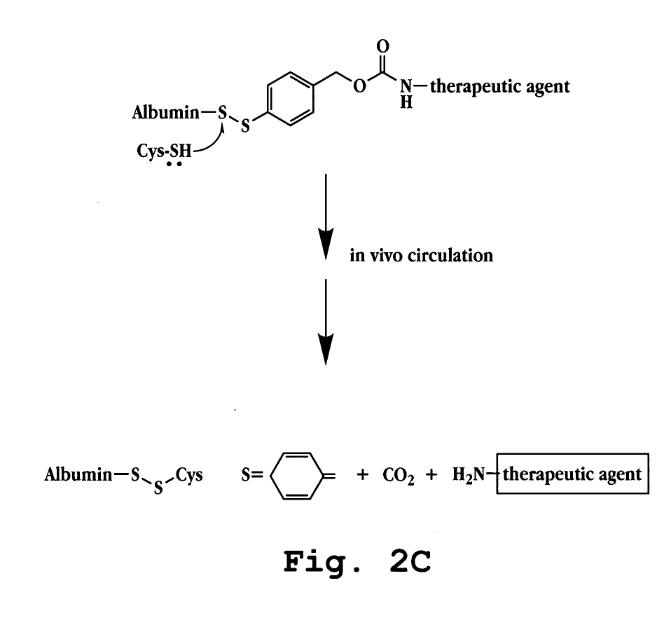

A general reaction scheme for preparation of a polymer-DTB-therapeutic agent conjugate is shown in FIG. 2A, with mPEG as the exemplary polymer. In general, a mPEG-DTB-leaving group compound is prepared according to method described in the art (see, Example 2A-2B of U.S. Pat. No. 6,605,299 incorporated by reference herein). The leaving group can be nitrophenyl carbonate as shown in FIG. 2A, or any other suitable leaving group. The mPEG-DTB-nitrophenyl carbonate compound is coupled to an amine moiety in a therapeutic agent by a urethane linkage. The R group on the carbon adjacent the disulfide in the compound can be H, CH₃, C₂H₅, C₃H₇, C₄H₉, (CₙH₂ₙ₊₁ in general with n=1-6) or the like and is selected according to the desired rate of disulfide cleavage. In addition, single or multiple PEG chains may be attached to a therapeutic agent by this chemistry to achieve a desired release profile, e.g. R can be a PEG residue. Reaction details for preparation of mPEG-methylDTB-therapeutic agent conjugates comprised of lysozyme and of erythropoietin as the therapeutic agents are given in Example 1. In the studies described herein, mPEG-MeDTB-therapeutic agent conjugates were used. That is, and with reference to FIG. 2A, the R group on the carbon adjacent the disulfide linkage was methyl. For ease of reference herein, this conjugate is simply referred to as mPEG-DTB-therapeutic agent.

When mPEG-DTB-therapeutic agent conjugate is exposed to plasma, the free thiol of albumin Cys-34 attacks the DTB moiety of the conjugate, resulting in its decomposition, as illustrated in FIG. 2B. The products of this process are free therapeutic agent, free mPEG, disulfide-linked mPEG-albumin, and albumin-therapeutic agent. The latter adduct is also disulfide-linked, as shown by release of the free therapeutic agent in the presence of small molecule thiols in plasma, as illustrated in FIG. 2C. Decomposition of the albumin-DTB-therapeutic agent after prolonged in vivo circulation yields the native therapeutic agent.

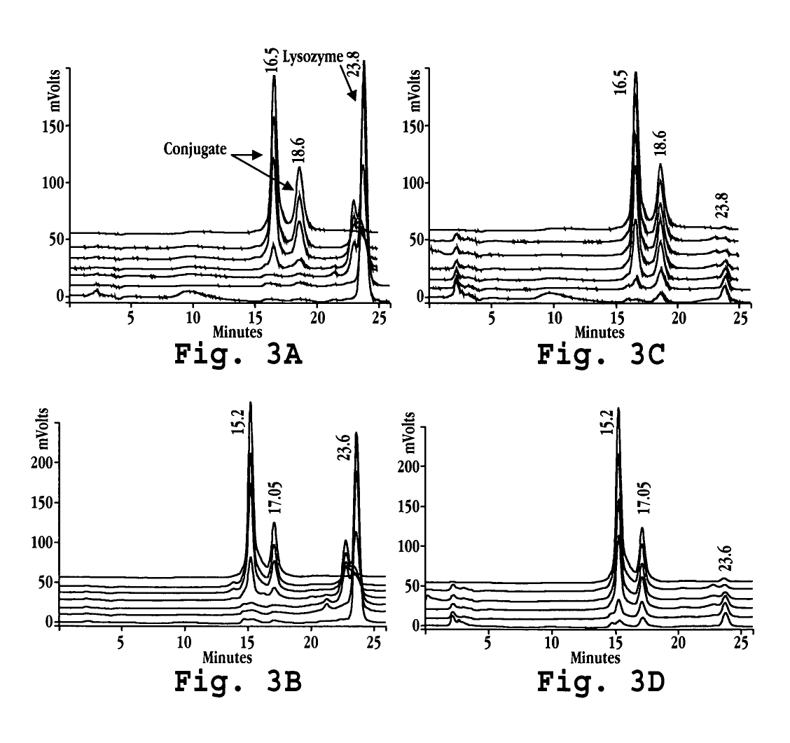

Example 2 describes a study to illustrate an embodiment of the method, where conjugates comprised of methoxypolyethylene glycol (mPEG) and of lysozyme as a model therapeutic agent were prepared. Synthesis of the mPEG-DTB-lysozyme conjugates is described in Example 1A and conjugates with mPEG molecular weights of 5 kDa and 12 kDa (designated herein as mPEG_{5K}-DTB-lysozyme and mPEG_{12K}-DTB-lysozyme, respectively) were prepared. The conjugates were incubated with cysteine or with bovine serum albumin for 47 hours. Aliquots were withdrawn at times of 10 minutes, 30 minutes, 2 hours, 6 hours, 23 hours, and 47 hours for analysis via HPLC (Example 2). The results are shown in FIGS. 3A-3D.

FIGS. 3A-3B are HPLC traces for conjugates of polymer-DTB-lysozyme incubated in cysteine for the various, indicated times (see the right hand side of FIGS. 3C, 3D). FIG. 3A shows the traces for mPEG_{5K}-DTB-lysozyme, and three peaks are observed, the peaks at 1.6 minutes and at 19 minutes corresponding to the conjugate and the peak at 24 minutes corresponding to the native protein lysozyme. The appearance of two peaks corresponding to the conjugate is likely a reflection of the position of the mPEG on the lysozyme since more than one isomeric form is possible and the various isomers will interact with the column differently. The increase in the peak corresponding to lysozyme with increasing incubation time, and the corresponding decrease in the conjugate peaks is apparent, consistent with continued cleavage of the conjugate with longer incubation time. FIG. 3B shows the traces for mPEG_{12K}-DTB-lysozyme. The increase in native free lysozyme at longer incubation times and a corresponding decrease in amount of conjugate is observed.

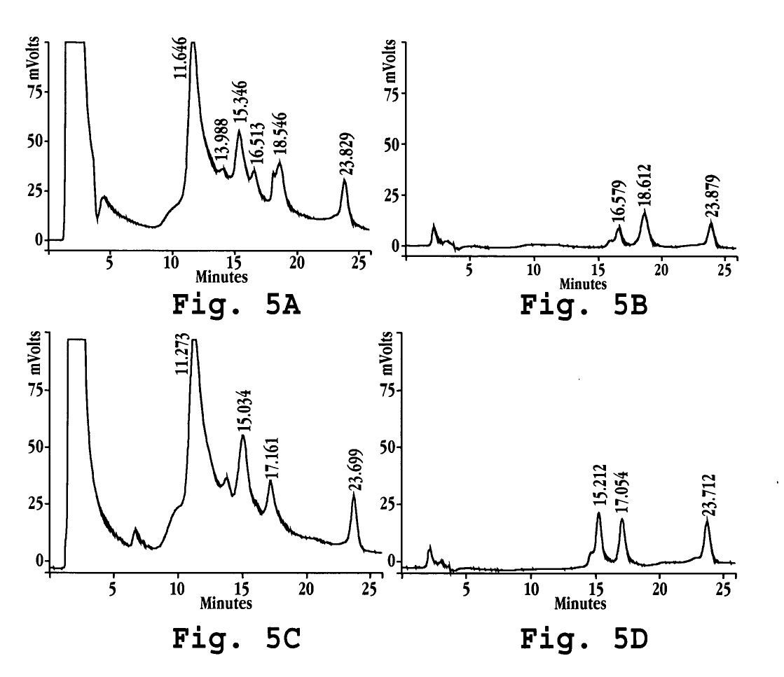

FIGS. 3C-3D are HPLC traces for conjugates of polymer-DTB-lysozyme incubated in bovine serum albumin (BSA) for various times between 10 minutes and 48 hours. FIG. 3C shows the traces for the mPEG_{5K}-DTB-lysozyme (1:1) conjugate. At early times in the incubation period, the peaks at 16.5 minutes and at 18.6 minutes corresponding to the conjugate are apparent. With increasing incubation in BSA, the appearance of a peak at 23.8 minutes is observed, corresponding to native, free lysozyme. Similar observations are made from the traces for the mPEG_{12K}-DTB-lysozyme conjugate (FIG. 3D). As shown in FIG. 5, discussed below, in these experiments the excess of albumin and albumin-containing adducts were removed by Q spin column. It is apparent that only a fraction of the PEG-DTB-lysozyme was converted to the free lysozyme by the BSA treatment.

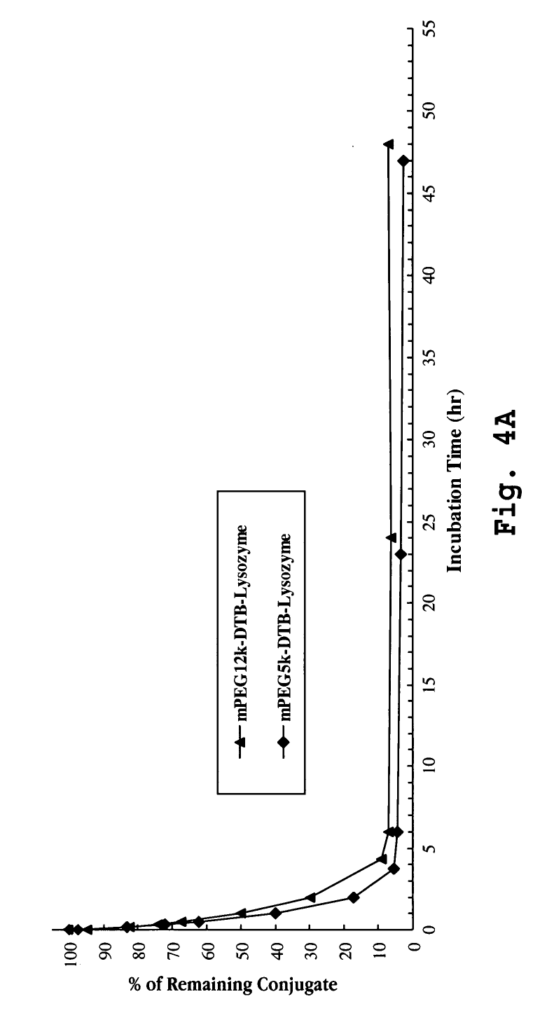

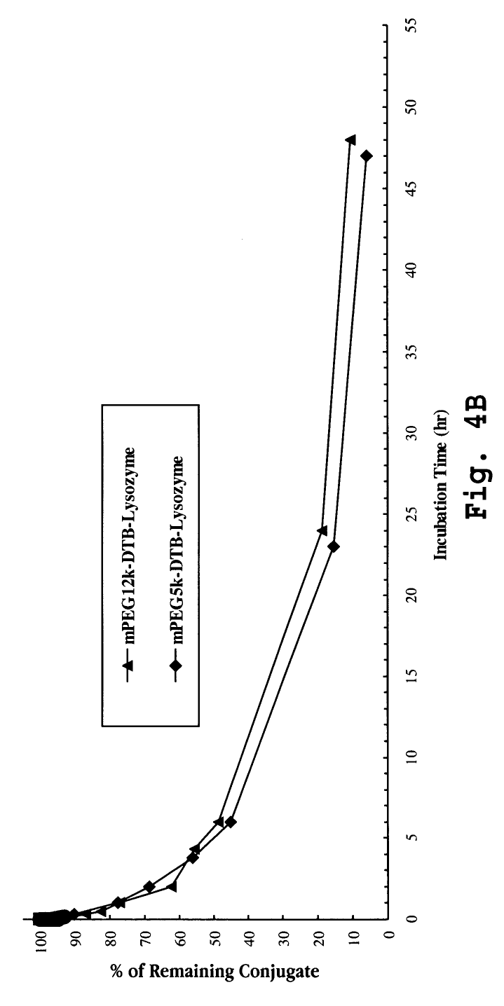

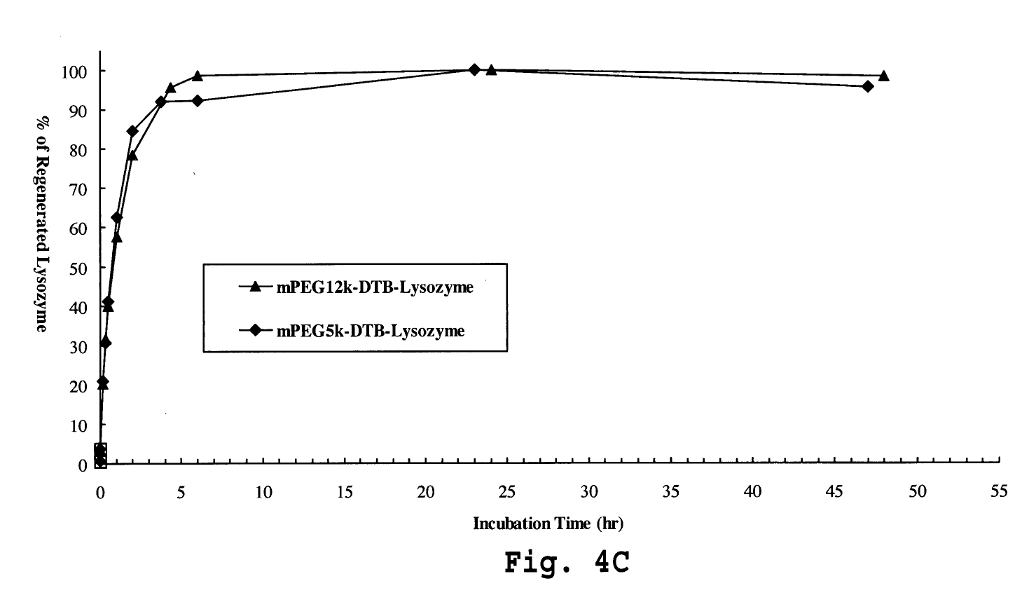

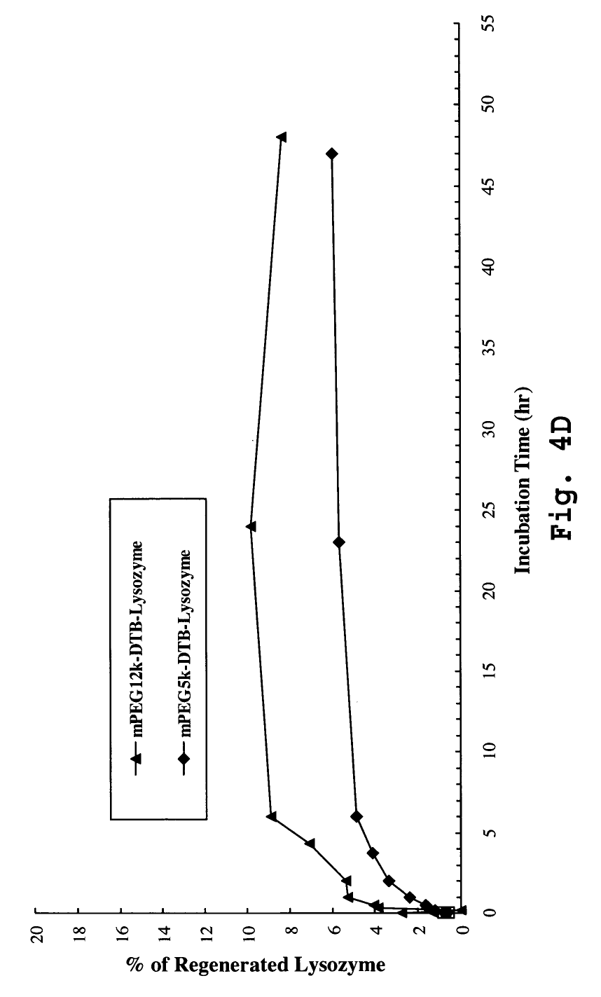

FIGS. 4A-4B are plots constructed from the HPLC traces showing the percent of remaining conjugate as a function of time upon incubation in cysteine (FIG. 4A) or in BSA (FIG. 4B). FIG. 4A shows the decrease in conjugate incubated with cysteine as a function of time, where the mPEG_{12K}-DTB-lysozyme conjugate (triangles) and the mPEG_{5K}-DTB-lysozyme conjugate (diamonds) had calculated half-lives of 60 minutes and 45 minutes, respectively.

FIG. 4B shows the decrease in remaining conjugates as a function of time, upon incubation in BSA. The slower decomposition of the conjugates relative to incubation in cysteine is apparent, and is also reflected in the calculated half-lives of 6 hours for the mPEG_{12K}-DTB-lysozyme conjugate (triangles) and 5 hours for the mPEG_{5K}-DTB-lysozyme conjugate (diamonds).

FIGS. 4C-4D are plots constructed from the HPLC traces showing the percent of regenerated lysozyme as a function of time upon incubation in cysteine (FIG. 4C) or in BSA (FIG. 4D). FIG. 4C shows that native, free lysozyme is regenerated from mPEG_{12K}-DTB-lysozyme conjugate (triangles) and the mPEG_{5K}-DTB-lysozyme conjugate (diamonds) over a period of 5-6 hours.

FIG. 4D shows the regeneration of native, free lysozyme from the conjugates upon incubation with BSA. Regeneration of the free protein is slower than regeneration of the conjugates with cysteine, with less than 10% of the protein regenerated in free form from either of the two mPEG-DTB-lysozyme conjugates.

The data in FIGS. 3-4 illustrate that both conjugates were cleaved by cysteine and by albumin. Cleavage by albumin did not fully regenerate free lysozyme as a result of the reaction with lysozyme and albumin. Thus, further studies were done to identify the presence and quantity of the albumin-lysozyme conjugate. In the HPLC analysis described above, the samples were passed over a Q-spin column to trap BSA prior to separation of the sample on the chromatography column. To determine whether the albumin-lysozyme conjugate was removed on the Q-spin column, samples that were not passed over a Q-spin column were analyzed by HPLC (CM-column) and the traces are shown in FIGS. 5A-5D. FIGS. 5A-5B correspond to the traces for the mPEG_{5K}-DTB-lysozyme conjugate incubated at room temperature in 4% BSA for 24 hours before (FIG. 5A) and after (FIG. 5B) passing the sample over a Q-spin column. Comparison of the traces shows the presence of a major peak at 11.6 minutes and a smaller peak at 15.3 minutes (FIG. 5A) that are not observed after the sample passes over the Q-spin column (FIG. 5B). The same observation is made for the conjugates of mPEG_{12K}-DTB-lysozyme (FIGS. 5C-5D). After cleaving the three mPEG-DTB-lysozyme conjugates with BSA, two new peaks at about 11 minutes and 15.2 minutes appear, along with the BSA peak in the first minutes of elution. The peaks at 11 minutes and 15.2 minutes had been previously eliminated after passing the samples through the Q spin columns.

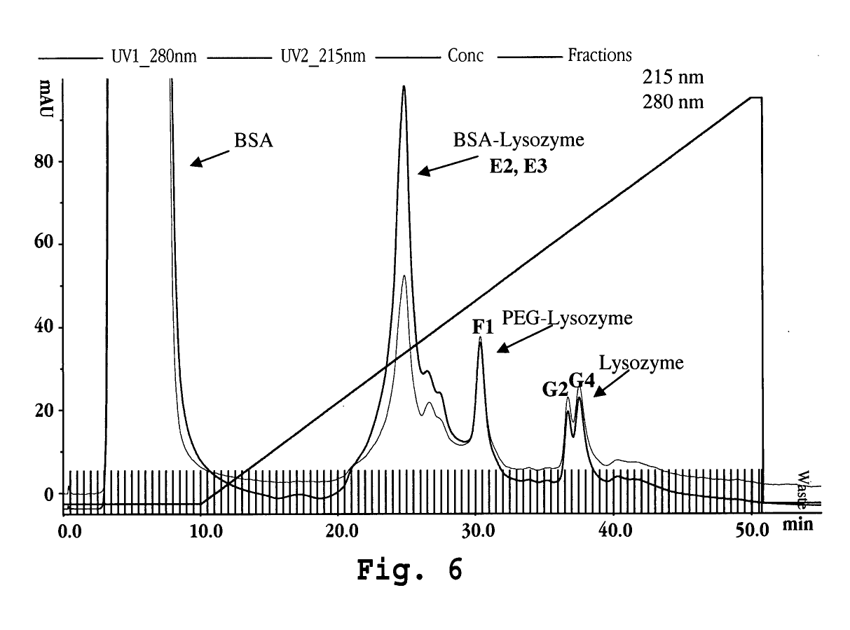

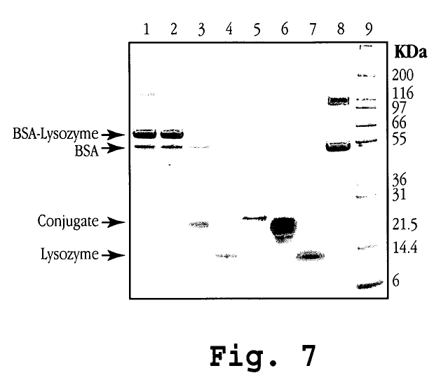

In a study designed to identify the newly formed peaks, described in Example 3, a 1:1 conjugate of mPEG_{5K}-DTB-lysozyme was prepared. The conjugate was incubated with BSA for two days and the incubation mixture was then analyzed by HPLC and by MALDI-TOFMS. The HPLC trace is shown in FIG. 6 and shows a peak corresponding to BSA early in the elution profile. Another peak occurs at about 24 minutes, identified as fractions E2, E3 and believed to correspond to albumin-lysozyme. The peak at about 30 minutes is identified as elution fraction F1, and the peaks at 37 minutes and 39 minutes are identified as elution fractions G2 and G4. These elution fractions were analyzed by SDS-PAGE, as will be discussed with respect to FIGS. 7-8.

Fractions obtained by ion-exchange chromatography (HPLC shown in FIG. 6) were analyzed by SDS-PAGE. The gel is shown in FIG. 7, where Lane 1 corresponds to the fraction identified as E2 on the HPLC trace of FIG. 6; Lane 2 corresponds to the fraction identified as E3 on the HPLC trace of FIG. 6; Lane 3 corresponds to the fraction identified as F1 on the HPLC trace, and appears to be the same as the main component of the mPEG5K-DTB-Lysozyme conjugate (lane 6); Lane 4 corresponds to the fraction identified as G2 on the HPLC trace of FIG. 6; Lane 5 corresponds to the fraction identified as G4 on the HPLC trace of FIG. 6; Lane 6 corresponds to the mPEG_{5K}-DTB-lysozyme (predominantly 1:1) conjugate; Lane 7 corresponds to lysozyme; Lane 8 corresponds to BSA; and Lane 9 is molecular weight markers.

The BSA migration on SDS gels corresponds to molecular weight of approximately 55 kilodaltons (kDa) (Lane 8), although the theoretical molecular weight of albumin is 66.5 kDa. Fractions E2 and E3 (Lanes 1, 2) contained a major band having a molecular weight of approximately 60 kDa. The anticipated migration of an albumin-lysozyme (theoretical molecular weight 81 kDa) product would be 69 kDa, the sum of BSA (55 kDa) and lysozyme (14 kDa). The fractions loaded onto Lanes 1 and 2 having a molecular weight of 65 kDa are in good agreement with the molecular weight for an albumin-lysozyme conjugate. Fraction F1 (Lane 3) contains mPEG-lysozyme conjugate and some BSA contaminant. Fraction G2 (Lane 4) contains lysozyme only. Fraction G4 (Lane 5) contains lysozyme and another band that appears to be of approximate molecular weight of 24 kDa.

When the fractions identified from the HPLC E2, G2, and G4 were analyzed by both reducing (with β-mercaptoethanol) and non-reducing SDS-PAGE the following picture emerged. The gel is shown in FIG. 8. Lane 1 corresponds to lysozyme with a molecular weight of 14 kDa. Lanes 2 and 3 correspond to mPEG-DTB-lysozyme conjugate (Lane 2) and the conjugated treated with β-mercaptoethanol (Lane 3). The β-mercaptoethanol reduced the conjugate, releasing the lysozyme from the mPEG-DTB adduct. Lanes 4 and 5 correspond to BSA (Lane 4) and BSA treated with β-mercaptoethanol (Lane 5). The BSA reduced with β-mercaptoethanol showed a shift in the molecular weight from nominal 55 kDa to 66 kDa (Lanes 4, 5), consistent with the real molecular weight of albumin. Fraction E2 (Lane 6) was decomposed into a lysozyme band and BSA bands (Lane 7) after treatment with β-mercaptoethanol. This thiolytic reduction was an indication that E2 contained lysozyme-albumin adduct linked by a disulfide-type bond. Fraction G2 (Lane 8) appeared to be unaffected by β-mercaptoethanol (Lane 9). Fraction G4 (Lane 10) was reduced to a single band (Lane 11) by β-mercaptoethanol, suggesting that the band at approximately 24 kDa (lane 10) was a lysozyme dimer (theoretical mol. weight approx. 28 kDa) that formed through a disulfide bond. Lane 12 shows the molecular weight markers.

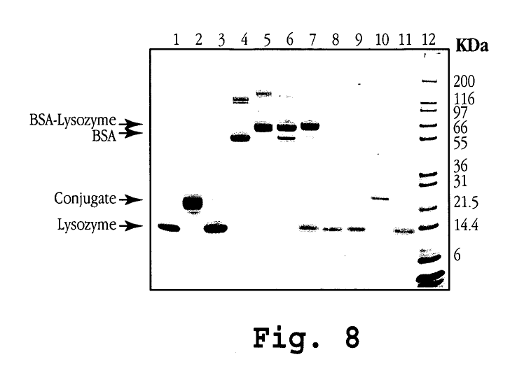

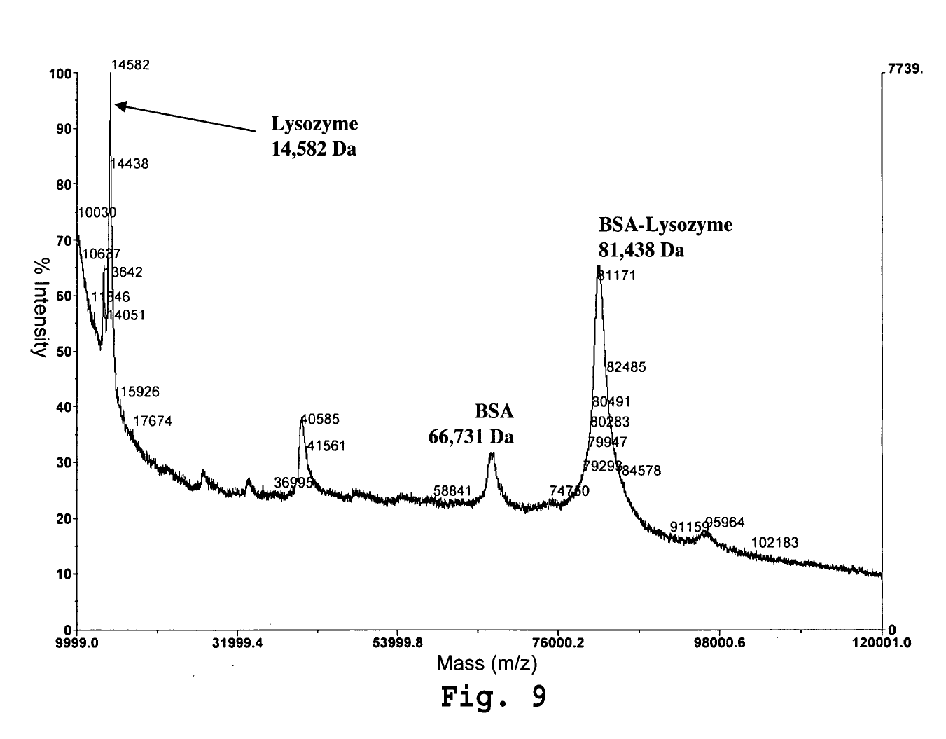

FIG. 9 shows the MALDI-TOFMS spectra of purified fraction E2 discussed with respect to FIG. 6. The signal at 14,582 corresponds to native, free lysozyme, which has a theoretical molecular weight of 14,388 Daltons. The peak at 66,731 corresponds to BSA, which has a molecular weight of 66,500 Daltons. The peak at 81,438 corresponds to a conjugate of albumin-lysozyme adduct, which has a theoretical molecular weight of 81 kDa. Note that under MALDI conditions disulfide linkages are often partially broken. Additional signals at 40585 and 95984 correspond to doubly charged albumin-lysozyme species and albumin-(lysozyme)₂ correspondingly.

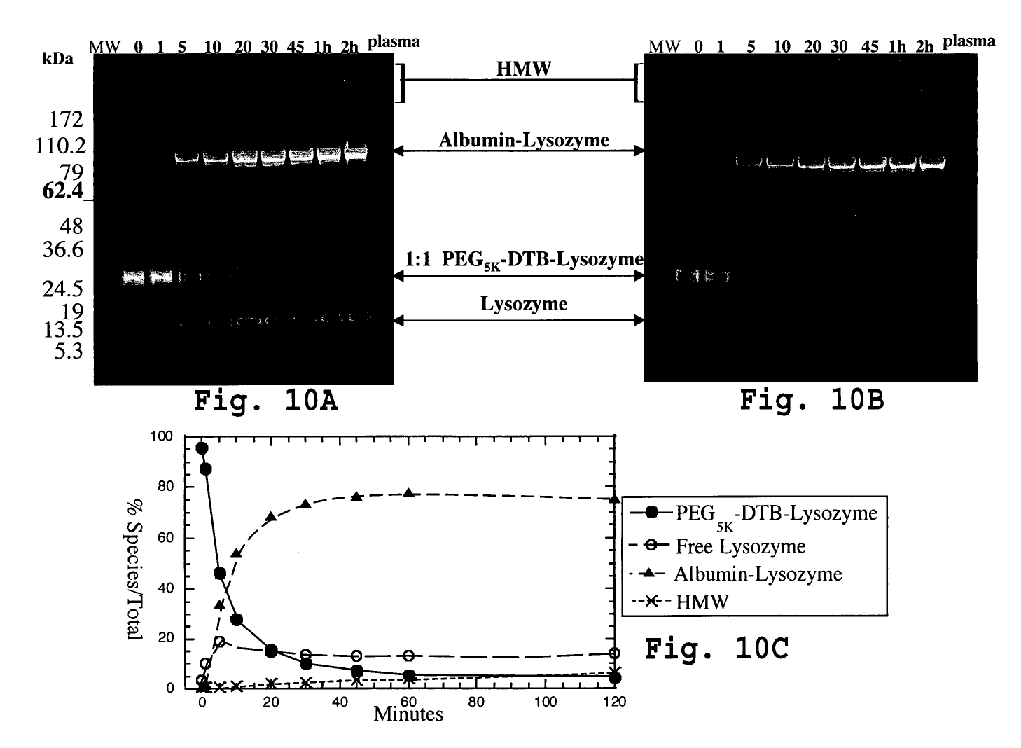

mPEG-DTB-lysozyme conjugates were also fluorescently labeled and examined in the presence of rat plasma or bovine serum albumin (BSA) over a timecourse at 37° C. As detailed in Example 4, the conjugates were labeled with ALEXA FLUOR 488, which labels free lysine residues in the lysozyme, and then incubated with rat plasma or with bovine serum albumin. Samples were collected as a function of time and analyzed by SDS PAGE. The fluorophore image was quantitated using a fluorescence imager. The SDS gel was also stained with SYPRO red to visualize total protein. The results are shown in FIGS. 10-13.

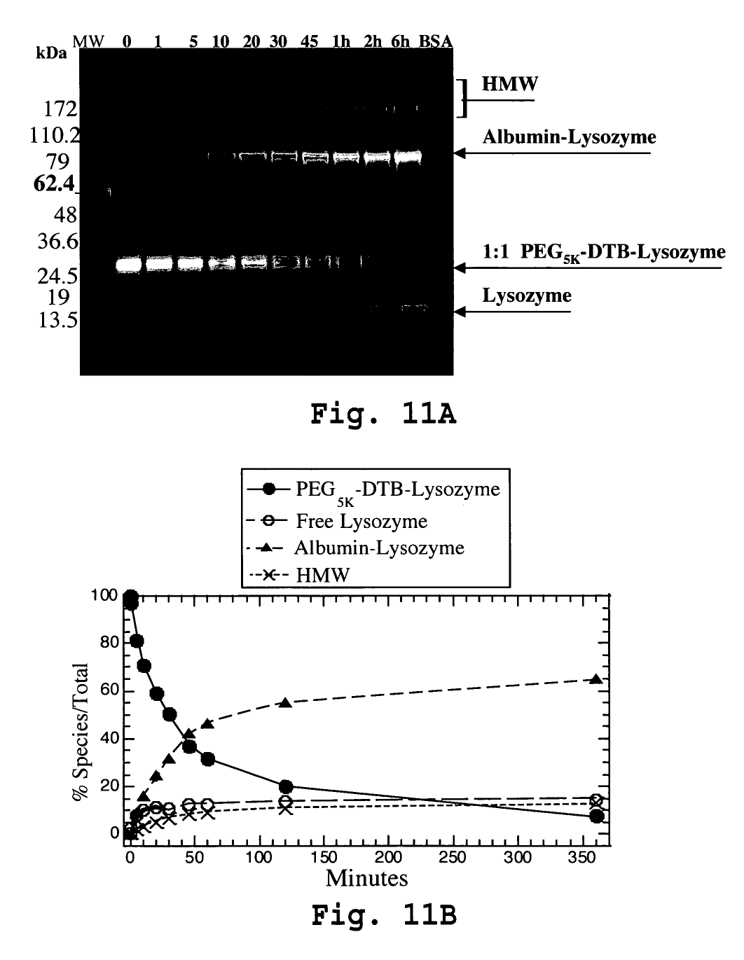

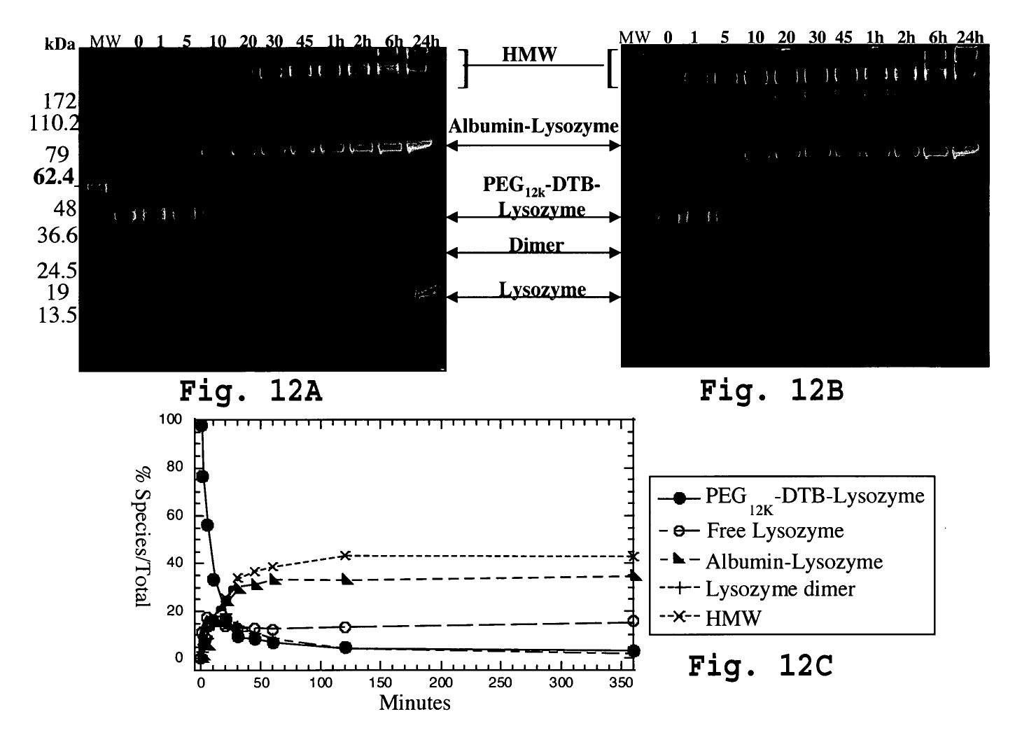

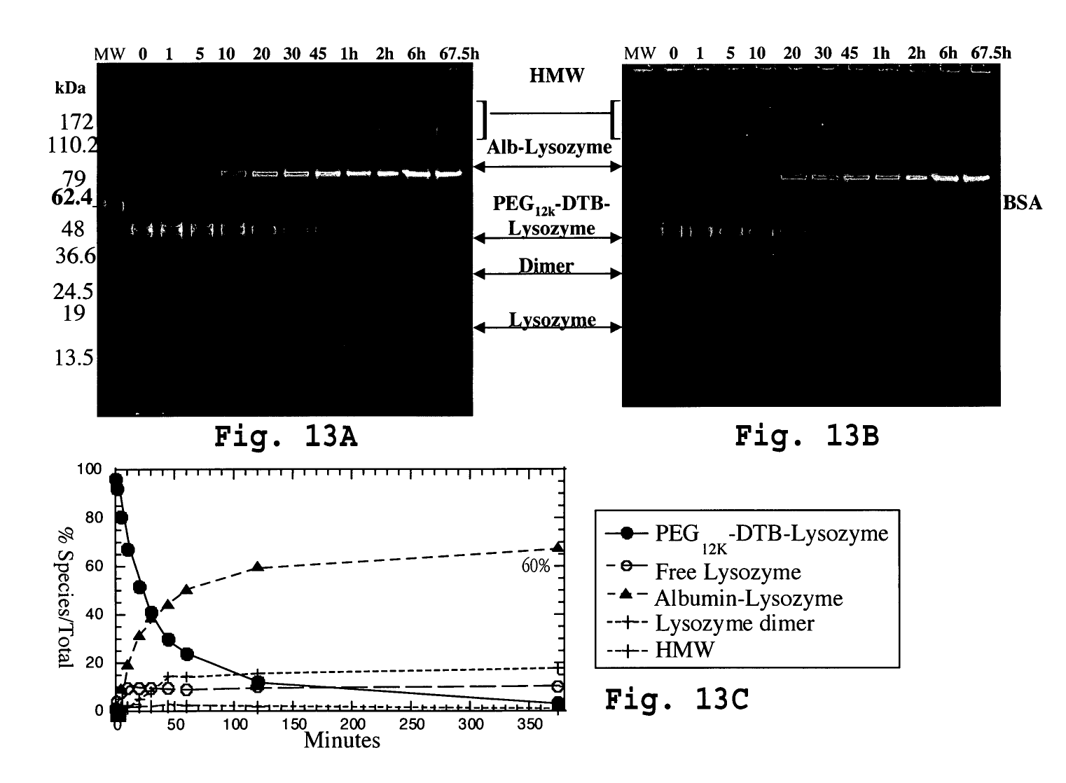

The data in FIGS. 10-13 shows that both mPEG_{5K}-DTB-lysozyme and mPEG_{12K}-DTB-lysozyme were converted to albumin-lysozyme and free lysozyme faster in the presence of plasma (FIGS. 10, 12) as compared to in the presence of bovine serum albumin (FIGS. 11, 13). This may be due in part to the presence of small molecule thiols in plasma. These studies also show that BSA alone as a cleaving agent was unable to yield the same extent of free lysozyme as rat plasma. The formation of a lysozyme dimer intermediate was not as separable for mPEG_{5K}-DTB-lysozyme (FIGS. 10, 11) as for mPEG_{12K}-DTB-lysozyme (FIGS. 12, 13), and therefore was included in the quantitation of mPEG_{5K}-DTB-lysozyme. High molecular weight (HMW) fluorescent species were observed, and were most prevalent for mPEG_{12K}-DTB-lysozyme incubated in plasma. The HMW species evidently result from interactions of the fluorescent conjugate with plasma proteins or albumin and are apparently not non-specific transfer of fluorophore. Also, these HMW species are cleaved from fluorescent lysozyme in the presence of reducing agent.

With respect to FIGS. 10C, 11B, 12C, and 13C, the data are expressed as the percent of each species relative to the total fluorescently-labeled material in each lane of the respective SDS-PAGE gel (FIGS. 10A, 11A, 12A, and 13A). Both the disappearance of mPEG-DTB-lysozyme conjugate (filled circles) and appearance of albumin-lysozyme (triangles) were observed. In addition, the appearance of free lysozyme (circles) was also observed. High molecular weight (HMW) fluorescent species (x symbols) were also formed upon incubation with rat plasma or bovine serum albumin. As seen in FIGS. 12C and 13C, an intermediate lysozyme dimer form was also quantitated (cross symbols).

The studies described above using lysozyme as a model therapeutic agent illustrate formation of a prodrug conjugate of albumin-lysozyme, subsequent to administration of a polymer-DTB-lysozyme conjugate. In a preferred embodiment, at least about 35% of the polymer-DTB-therapeutic agent conjugate that is administered is converted to a prodrug conjugate comprised of endogenous albumin and the therapeutic agent. In other words, of the total amount of therapeutic agent administered in the form of a polymer-DTB-therapeutic agent conjugate, at least about 35%, more preferably at least about 50%, still more preferably at least about 70%, is found in the blood two hours after administration in the form of an albumin-therapeutic agent conjugate.

Additional studies were conducted using erythropoietin (Epo) as a model therapeutic agent. A conjugate comprised of mPEG_{12K}-DTB-Epo was prepared, as described in Example 5. For comparison, a non-cleavable conjugate of mPEG-Epo was also prepared. The conjugates were incubated in the presence of human serum albumin. In order to ensure all reaction products were visualized by SDS-PAGE, the concentration of HSA was significantly lower than physiological conditions and small molecule thiols were not included in the reaction, to prevent subsequent cleavage of the newly formed albumin-Epo conjugates. The albumin-Epo product is generated through a thiolytically cleavable bond as was observed when the reaction was treated with cysteine (data not shown).

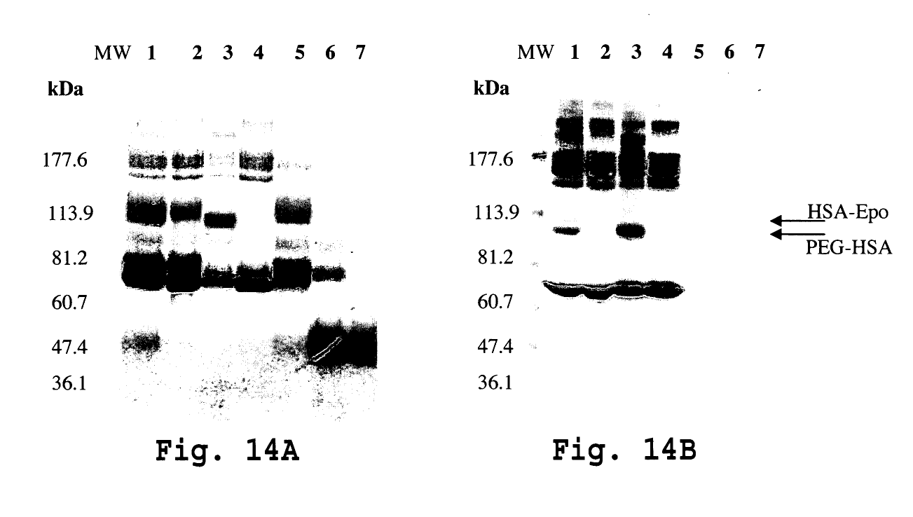

FIG. 14A shows the SDS-PAGE gel of conjugate products where Lane 1 shows the mPEG_{12K}-DTB-Epo conjugate in the presence of HSA and Lane 2 shows the mPEG_{12K}-Epo non-cleavable conjugate in the presence of HSA. Lane 3 corresponds to HSA incubated with excess conjugate of mPEG_{12K}-DTB-glycine. Lane 4 shows HSA alone and Lane 5 shows the mPEG_{12K}-DTB-Epo conjugate alone. Lane 6 corresponds to the mPEG_{12K}-DTB-Epo conjugate incubated with 2 mM cysteine. Lane 7 is Epo alone. These experiments demonstrate that the attachment of albumin to erythropoietin is dependent on the presence of the cleavable mPEG-DTB linker. Neither Epo alone nor the noncleavable mPEG-Epo formed the albumin-Epo conjugate. Further, the albumin-Epo conjugate itself was not PEGylated in the process of the albumin-Epo formation. This indicates that the conversion to albumin-Epo requires the removal of PEG-DTB moiety. This evidence is consistent with cleavage of PEG occurring prior to or simultaneously with an attachment of albumin via the thiobenzyl linker moiety of mPEG-DTB (FIG. 2B).

According to prestained molecular weight markers in the gels, the apparent molecular weights of the molecules of interest by SDS-PAGE are as follows:

TABLE 1

| | |
|---|---|
| Albumin-Epo | 111 kDa |
| 2:1 mPEG_{12K}-DTB-Epo | 105 kDa |
| mPEG_{12K}-Albumin | 96 kDa |
| 1:1 mPEG_{12K}-DTB-Epo | 75 kDa |
| Albumin | 66 kDa |
| Epo | 45 kDa |

FIG. 14B is an immunoblot probed with anti-HSA where Lanes 1-7 correspond to the same samples in the SDS-PAGE gel of FIG. 14A. The albumin-Epo conjugate is visible at about 111 kDaltons, as indicated by the arrow labeled “HSA-Epo” in the drawing. The mPEG-albumin conjugate is also visible, and is indicated in the drawing by the arrow labeled “PEG-HSA”. To confirm the identity of an mPEG-albumin conjugate at 96 kDa and of PEG-EPO at 105 and 75 kDa, iodine PEG staining and an antibody to EPO were used (data not shown). The position of mPEG_{12K}-DTB-albumin was verified by the control reaction (sample in Lane 3) of albumin with mPEG_{12K}-DTB-glycine.

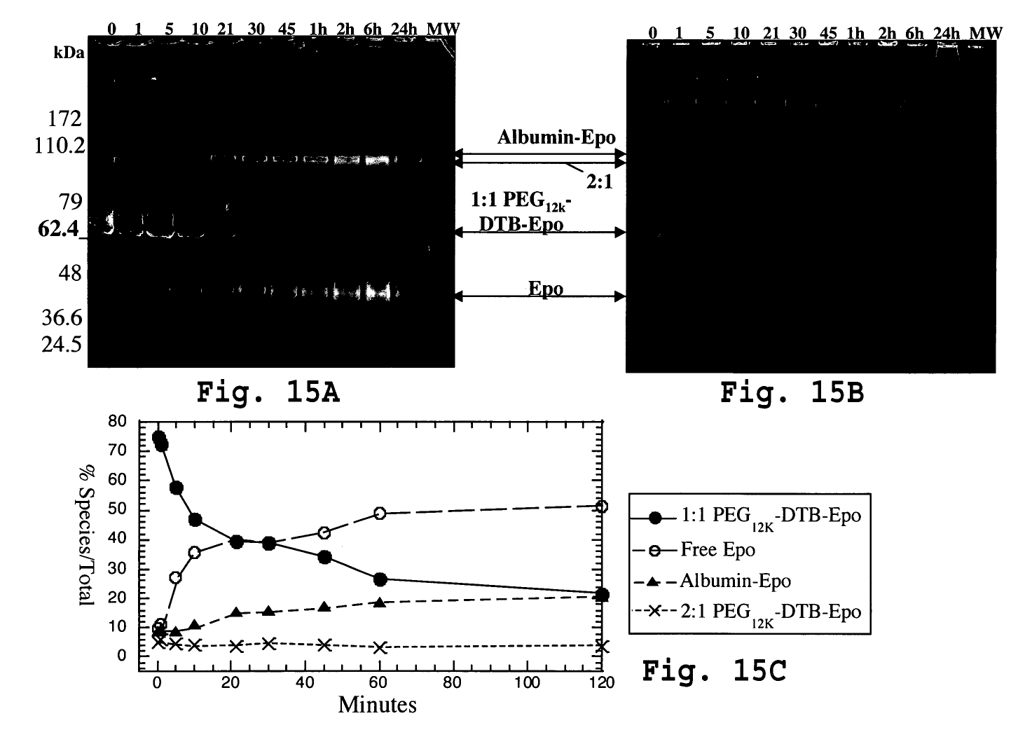

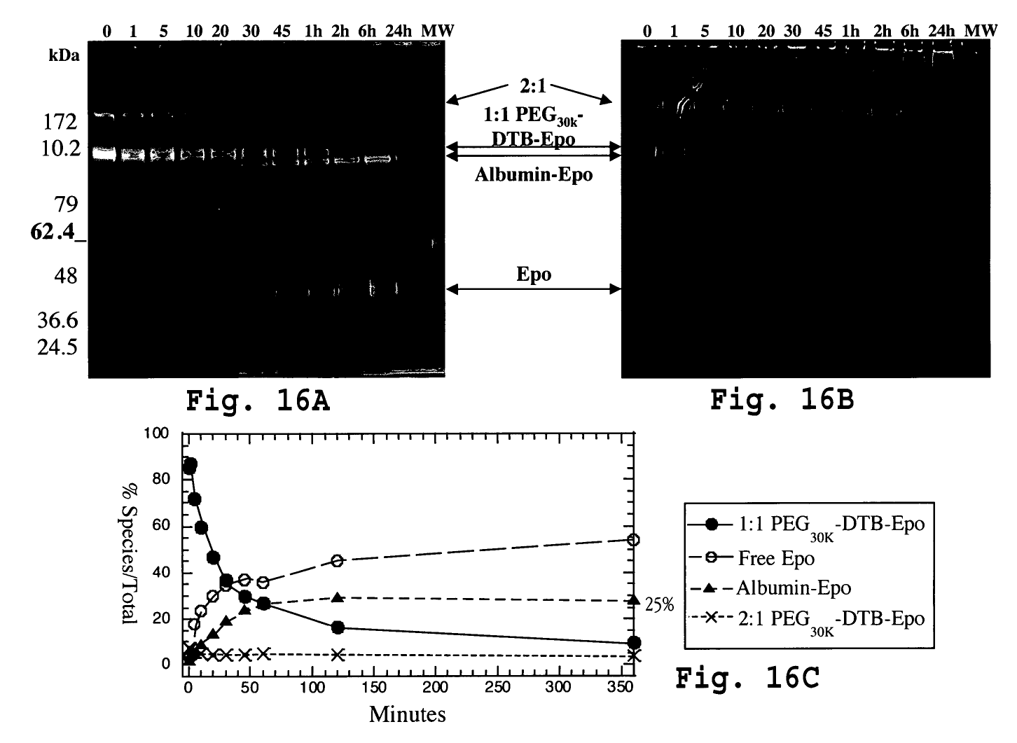

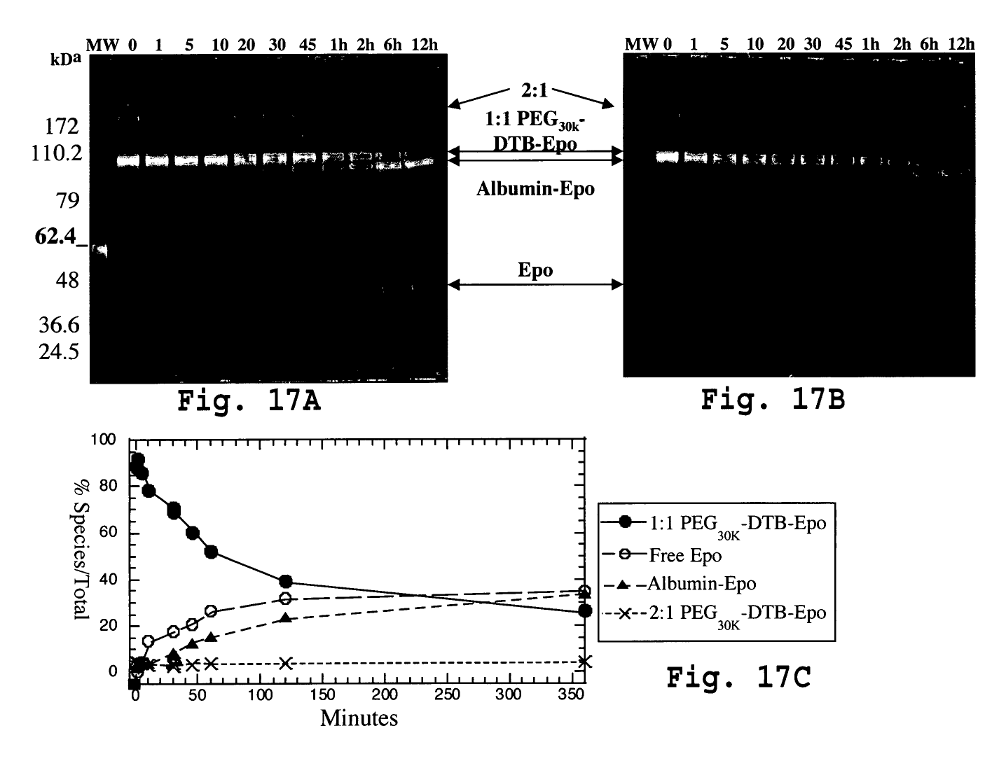

Fluorescently-labeled mPEG-DTB-Epo conjugates were observed in the presence of rat plasma or bovine serum albumin over a timecourse at 37° C., similar to the study discussed above for the mPEG-DTB-lysozyme conjugates (Example 4). The data for the mPEG-DTB-Epo conjugates (mPEG molecular weights of 12 kDa and 30 kDa) is shown in FIGS. 15-17. Identification of albumin-containing bands was confirmed by immunoblot as in FIG. 14B. For mPEG_{12K}-DTB-Epo (FIGS. 15A-15C), the overlap of 2:1 mPEG_{12K}-DTB-Epo with albumin-Epo obscured the quantitation of these species, so mPEG_{30K}-DTB-Epo was utilized to clarify this. A comparison of FIG. 15C and FIG. 16C shows that a longer (higher molecular weight) mPEG chain slows the rate of cleavage of the disulfide linkage in the mPEG-DTB-Epo conjugate. FIGS. 15B, 16B, and 17B show total protein content, visualized by staining with SYPRO red. Trace amounts of mPEG-disulfide-protein conjugates at a greater substitution ratio than 1:1 were also observed (2:1 polymer:protein).

The data in FIGS. 15C, 16C, and 17C are expressed as the percent of each species out of the total fluorescently-labeled material in each lane of the respective gel (FIGS. 15A, 16A, 17A). The disappearance of mPEG-DTB-Epo protein conjugate (filled circles) and appearance of albumin-Epo (triangles) were observed. In addition, the appearance of free Epo (circles) was also observed. Cleavage of the conjugate in plasma yielded a faster rate of cleavage than in bovine serum albumin.

Notably, and in comparison to the data described above on the lysozyme-containing conjugates, only about 25% of the Epo in the form of an mPEG-DTB-Epo conjugate was converted into an albumin-Epo conjugate, considerably less than observed for the lysozyme conjugates. Incubation of mPEG-DTB-Epo conjugate in plasma for two hours and longer resulted in 25-30% of the Epo appearing in the plasma in the form of an Epo-albumin conjugate.

Table 2 is a summary of the cleavage rates (T_{1/2} values) determined from the data presented in FIGS. 10-13 and FIGS. 15-17. These rates represent the time (in minutes) for decomposition of half of the initial amount of PEG-DTB-protein present at time zero after treatment with rat plasma or bovine serum albumin.

TABLE 2

| Conjugate | T_{1/2}(min.) Rat Plasma | T_{1/2}(min.) BSA* |
|---|---|---|
| PEG_{5K}-DTB-Lysozyme | 5.2 | 31 |
| PEG_{12K}-DTB-Lysozyme | 6.4 | 23 |
| PEG_{12K}-DTB-Epo | 34 | — |
| PEG_{30K}-DTB-Epo | 23 | 98 |

*BSA = bovine serum albumin

The blood circulation half-life of the PEG_{12K}-DTB-lysozyme conjugate was about five-fold less than the blood circulation half-life of the PEG_{12K}-DTB-Epo conjugate, indicating a faster rate of cleavage of the disulfide linkage and formation of a conjugate with albumin.

The results above for the conjugates prepared with the model proteins Epo and lysozyme shows that an albumin-protein conjugate is formed when a polymer-DTB-protein conjugate interacts with albumin, with the smaller molecular weight protein yielding a greater amount of albumin-protein conjugate. Potentially, hindrance caused by the therapeutic protein charge or structure near the site of DTB attachment contributes to the yield of albumin-protein conjugate formed. The studies also show that the albumin-protein conjugate is cleaved in the presence of a reducing thiolytic agent, indicating that the linker is disulfide, likely to be the thiobenzyl linker.

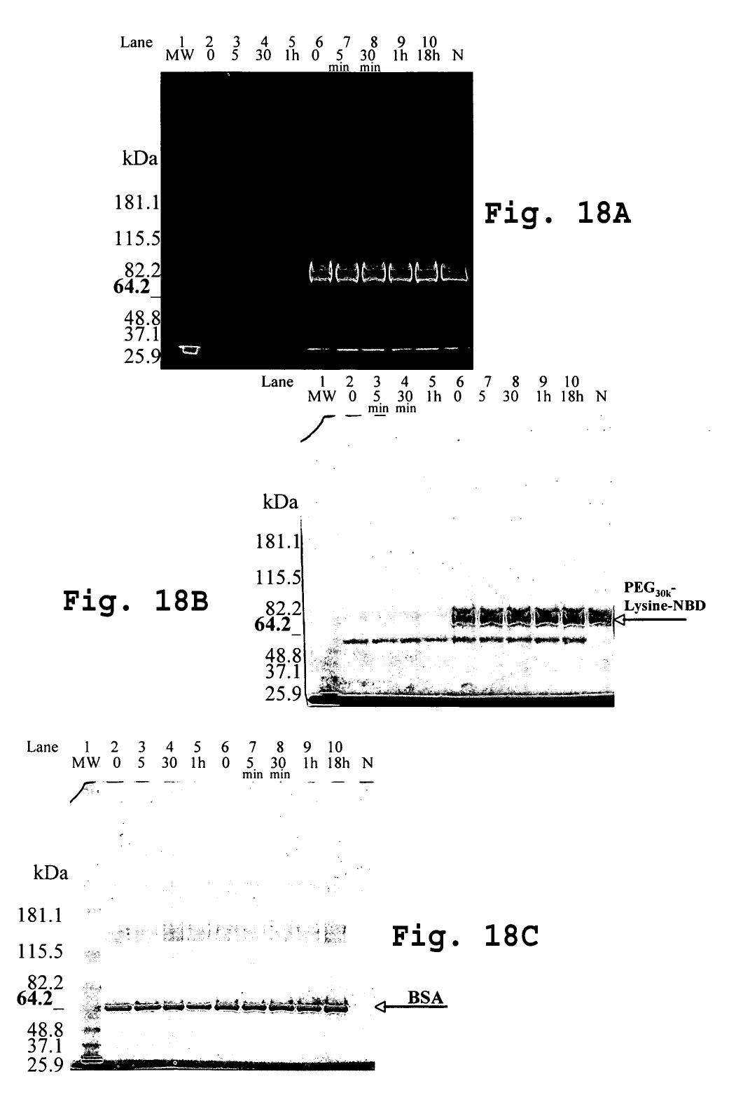

Additional studies examining the cleavage rate of the disulfide-linker were performed, as described Example 6. Rather than a protein as in Examples 4 and 5, a small molecule, fluorescent amino acid derivative, lysine-NBD (7-nitrobenz-2-oxa-1,3-diazole), having a molecular weight of 344.79 Daltons, was used. Briefly, mPEG_{30K}-DTB-NPC was conjugated to the fluorescent lysine-NBD. As a control, a non-cleavable conjugate of MPEG and lysine-NBD was prepared using mPEG-succinimidyl carbonate. The conjugates were incubated in bovine serum albumin with aliquots withdrawn at specified times for analysis by SDS-PAGE. The gels are shown in FIGS. 18A-18C. In all of FIGS. 18A-18C, Lanes 2-6 correspond to incubation of the non-cleavable mPEG-DTB-lysine-NBD conjugate with an equimolar concentration of BSA for 0 minutes, 5 minutes, 30 minutes, and 1 hour. Lanes 6-10 correspond to the incubation of the non-cleavable the mPEG-DTB-lysine-NBD conjugate with BSA, the conjugate present in a 10-fold higher concentration, for incubation times of 0 minutes, 5 minutes, 30 minutes, 1 hour, and 18 hours. The gels show that essentially no interaction occurs between BSA and the non-cleavable mPEG_{30K}-Lysine-NBD at equimolar or 10-fold PEG concentrations, 37° C. for the timecourse indicated. The PEG derivative alone is shown in FIG. 18A, lane N. FIGS. 18B and 18C show the same gel, but stained with iodine for detection of PEG (FIG. 18B) or with Coomassie blue stain, for protein visualization.

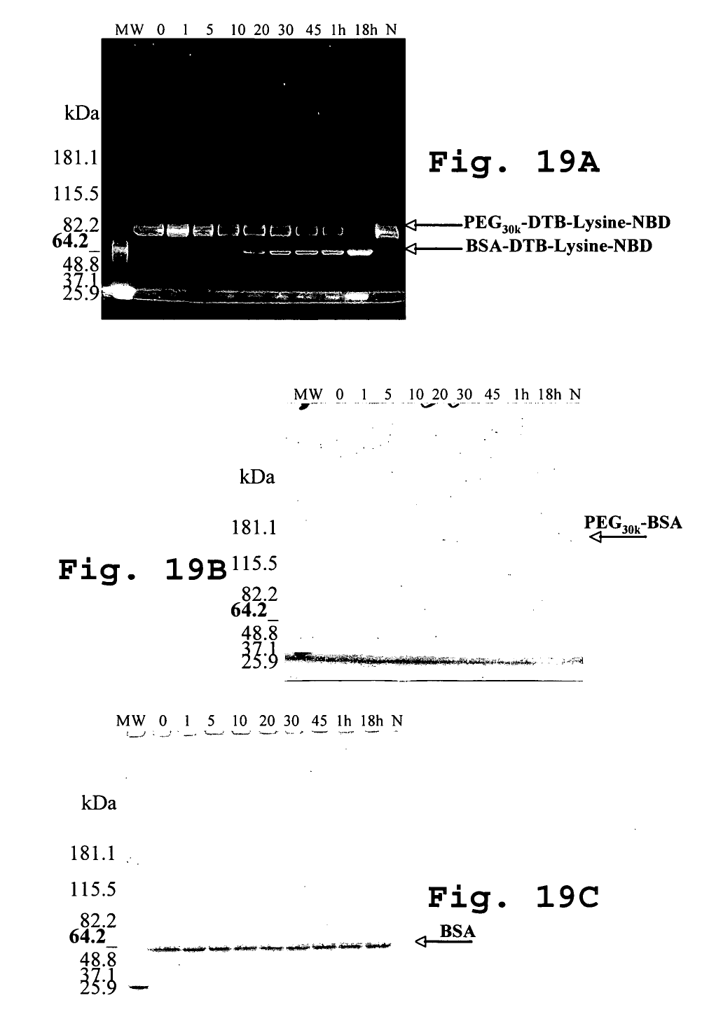

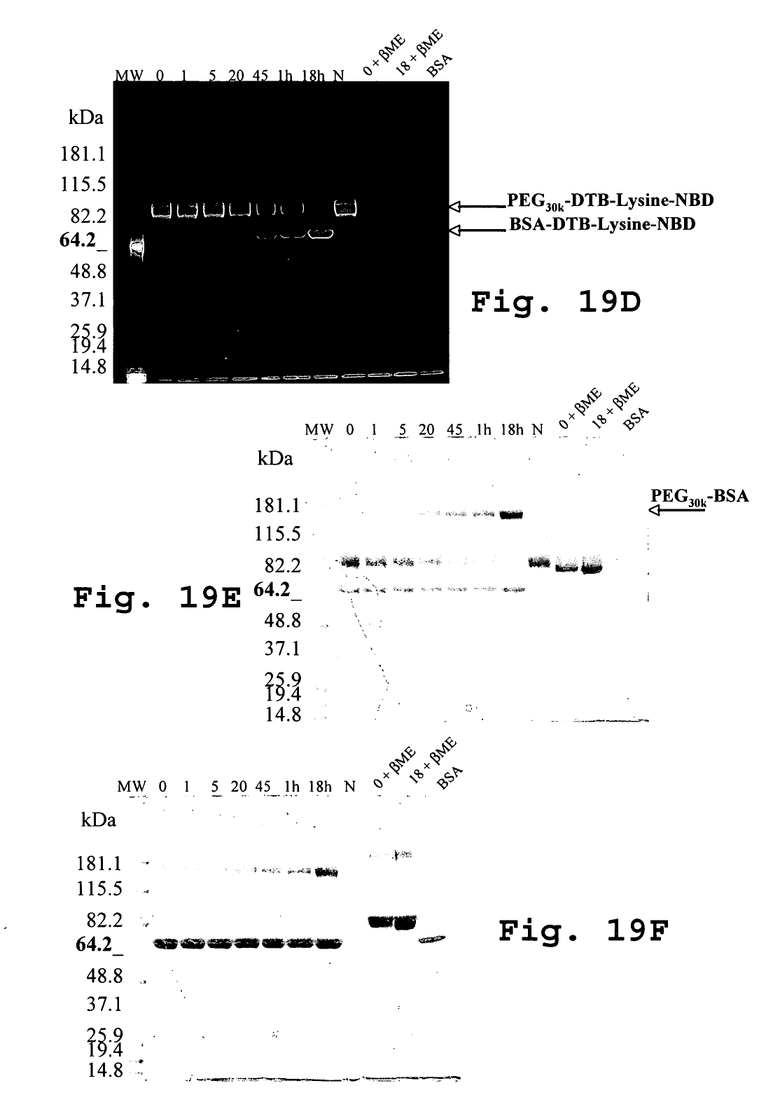

FIGS. 19A-19D are SDS-PAGE gels for the studies conducted with fluorescently-labeled mPEG_{30K}-DTB-Lysine-NBD incubated with an equimolar amount of BSA. FIGS. 19A-19C correspond to samples run on a non-reducing gel, Tris-acetate. FIGS. 19D-19F correspond to samples run on a conventional SDS-PAGE gel. The lanes in each gel correspond to the incubation time of the conjugate in BSA, as noted along the upper portion of each gel, with the molecular weight markers in the lane denoted MW and lane N (FIGS. 19A-19C) corresponding to mPEG_{30K}-DTB-Lysine-NBD alone. As seen, new adducts are formed and visible by SDS-PAGE within 5 minutes of incubation. BSA becomes fluorescently labeled, presumably with lysine-NBD, over the timecourse of the incubation period (FIGS. 19A, 19D). Also, by iodine staining for PEG, a band corresponding to mPEG_{30K}-BSA appears at approximately 126 kDa (FIGS. 19B, 19E) over time. For comparison, BSA alone is shown in FIG. 19D, Lane BSA, and mPEG_{30K}-DTB-Lysine-NBD alone in FIG. 19A, Lane N. In the presence of β-mercaptoethanol, the DTB linker of mPEG_{30K}-DTB-lysine-NBD is cleaved to yield mPEG₃OK and lysine-NBD (FIGS. 19D-19F). The formed adducts in the BSA reaction are also likely disulfide-linked as seen in previous Examples. A zero timepoint sample of the BSA reaction was treated with β-mercaptoethanol during gel sample preparation (FIG. 19D, Lane “0+αME”). Nearly complete cleavage of the DTB-linker was observed under these conditions. An 18 hour timepoint sample was treated the same way (FIG. 19D, Lane “18+βME”). The addition of reducing agent to the 18 hour timepoint may not have been adequate to fully cleave the BSA-DTB-Lysine-NBD adduct or it is possible an alternate mechanism for adduct formation also occurs. Note that reduced BSA and PEG_{30K} migrate about the same distance by SDS-PAGE. SDS-PAGE analysis cannot determine the identity of the fluorescent higher molecular weight NBD adducts migrating at >115 kDa (FIGS. 19A, 19D). Whether this is dimerized BSA in which one or both BSA molecules also become labeled with lysine-NBD or other higher molecular weight adducts (specific or non-specific) is not known, however, the signal from higher molecular weight NBD fluorescence is less than 5% of the total fluorescence.

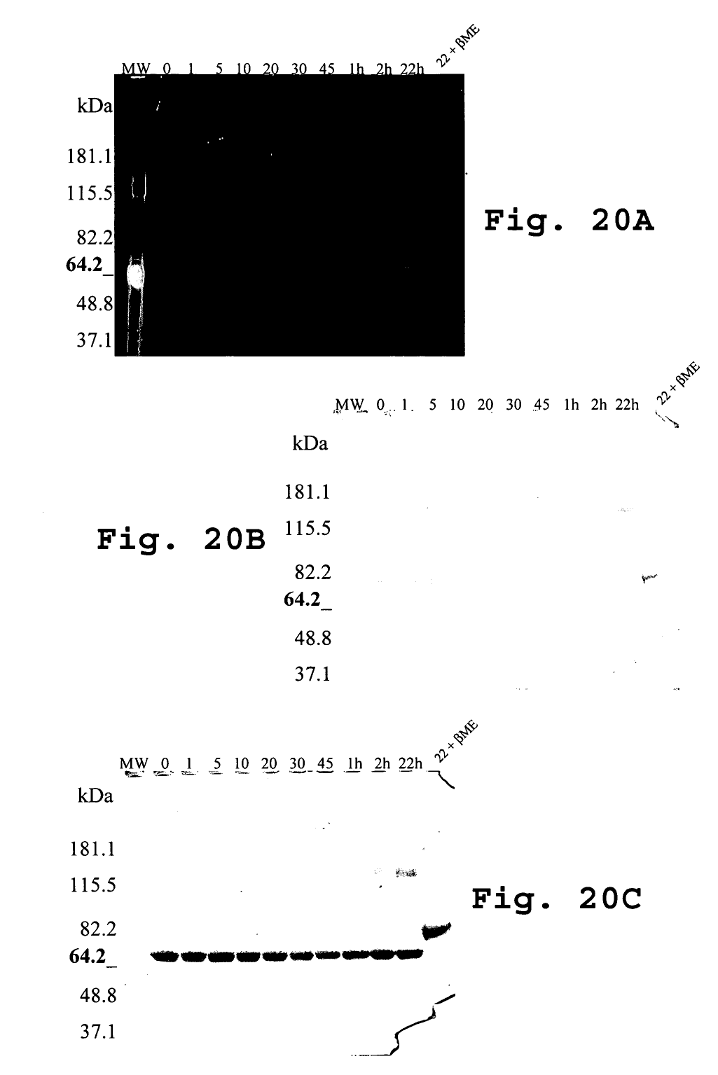

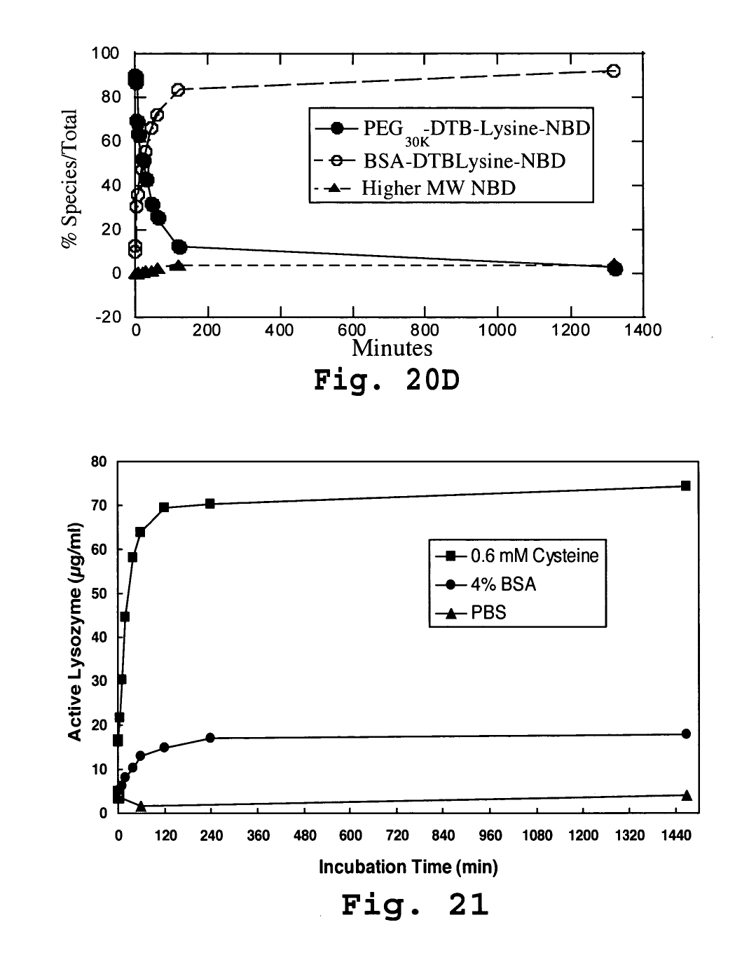

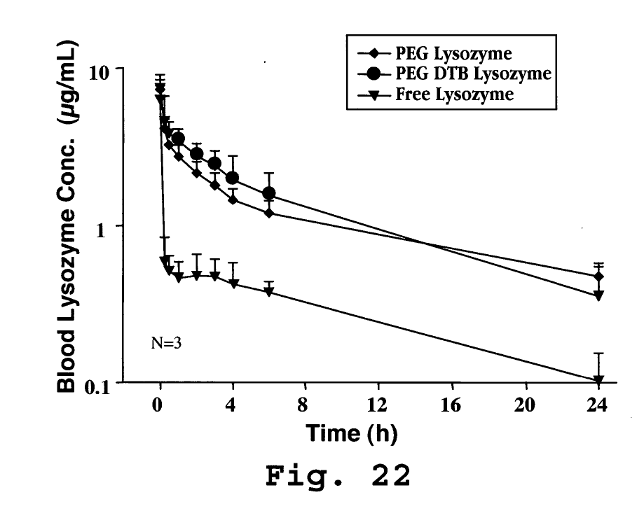

A similar study was conducted where mPEG_{30K}-DTB-Lysine-NBD conjugate was incubated with an equimolar concentration of BSA. The corresponding SDS-PAGE gels are shown in FIGS. 20A-20C and the quantitation of fluorescently-labeled lysine-NBD shown in FIG. 20D. With respect to the gels, FIG. 20A shows the samples as a function of incubation time, as indicated along the top of the gel. FIGS. 20B-20C correspond to the same gel, stained for PEG visualization and for protein visualization, respectively. The data in FIG. 20A was quantitated to yield the graph in FIG. 20D, with the exception of Lane 22+βME which was run in the presence βME. Both the disappearance of mPEG_{30K}-DTB-Lysine-NBD (FIG. 20D, filled circles) and the appearance of BSA-DTB-Lysine-NBD (FIG. 20D, open circles) were observed. The approximate time to half mPEG_{30K}-DTB-Lysine-NBD remaining was 27.5 min, less than a third of the time for decomposition of mPEG_{30K}-DTB-Epo (Table 2). The BSA-DTB-Lysine-NBD species formed was 92.2% of the total NBD signal by the assay endpoint.

In another study, described in Example 7, a Micrococcus luteus turbidity assay was used to analyze mPEG_{5K}-DTB-lysozyme activity after treatment with 4% BSA or cysteine, or with saline as a control. FIG. 21 shows the concentration of active lysozyme, in μg/mL, as a function of time, in minutes, when the mPEG_{5K}-DTB-lysozyme conjugate was incubated with cysteine (squares), BSA (circles), or saline (triangles). After cleavage with BSA, the active lysozyme concentration, by this assay, was approximately 18 μg/mL after 24 hours (circles). This amount is only 24% of the active lysozyme regenerated from the cysteine cleavage (74 μg/mL, squares). Thus, BSA treatment of mPEG-DTB-lysozyme resulted in formation of a BSA-lysozyme conjugate, since the BSA-lysozyme conjugate has no enzymatic activity whereas the cysteine cleaved mPEG-DTB-lysozyme conjugate resulted in release of active lysozyme. The data in FIG. 21 shows that the mPEG-DTB-lysozyme conjugate has little enzymatic activity (1-5%) and that incubation of the conjugate in saline at 37° C. for up to 24 hours did not induce release of the lysozyme from the PEG. The data also shows that the enzymatic activity is regenerated upon cysteine-mediated cleavage of mPEG-DTB-lysozyme, while only a fraction of active lysozyme is formed from BSA cleavage. This is consistent with formation of inactive albumin-lysozyme conjugate as the main product of the BSA reaction. The starting conjugate, mPEG-DTB-lysozyme, showed minimal activity in PBS over prolonged time.

In vivo administration of the polymer-DTB-therapeutic agent was studied by administering a conjugate comprised of mPEG_{12K}-DTB-lysozyme to rats. As described in Example 8, the mPEG_{12K}-DTB-lysozyme was administered intravenously to a group of three rats. Additional rats were treated with a noncleavable mPEG-lysozyme conjugate or with free lysozyme as comparative control. Blood samples were taken at selected intervals over a 24 hour time period and analyzed for lysozyme concentration. The results are shown in FIG. 22.

FIG. 22 shows the lysozyme concentration as a function of time (i.e., the pharmacokinetic profile) for the three treatment groups. The free lysozyme (inverted triangles) was cleared rapidly from the blood stream. Lysozyme administered in the form of a noncleavable mPEG-lysozyme conjugate (diamonds) or with mPEG_{12K}-DTB-lysozyme conjugate (circles) showed similar extended circulation lifetimes. The half-lives and AUC values for both the noncleavable mPEG-lysozyme conjugate and the cleavable mPEG_{12K}-DTB-lysozyme conjugate were similar. In vitro work has demonstrated that the polymer-DTB-drug conjugate is cleaved relatively rapidly in plasma and upon incubation in albumin solutions similar to conditions in vivo, due to the presence of reducing thiolytic agents. The comparable long circulation life of the cleavable mPEG_{12K}-DTB-lysozyme conjugate to the noncleavable mPEG-lysozyme conjugate is consistent with the formation of a long-circulation albumin-lysozyme product. Thus, the in vivo study supports that formation of an albumin-lysozyme adduct is the basis for the slow clearance and long circulation lifetime of the model drug (lysozyme).

From the foregoing, it can be seen how various objects and features of the invention are met. The polymer-disulfide-therapeutic agent conjugate that is prepared ex vivo can be administered to a subject to achieve formation of an albumin-therapeutic agent conjugate that has a long drug circulation lifetime. While the studies above use a dithiobenzyl linkage, it will be appreciated that other disulfide linkages are equally applicable. The therapeutic agent in its native form is recovered after thiolytic cleavage of the albumin-therapeutic agent conjugate in vivo. The albumin-therapeutic agent conjugate is formed in situ using endogenous albumin. The long circulation time of albumin, and thus of the albumin-therapeutic agent conjugate, provides the ability of targeting the drug to tissues, such as tumors or to the synovium for treatment of rheumatoid arthritis. Those of skill in the art can appreciate the variety of disease conditions that would benefit from an extended blood circulation lifetime of a therapeutic agent. By increasing the circulation time of therapeutics such as protein molecules, less therapeutic agent may be required for treatment, thus reducing costs per dose. In addition, less frequent dosing is possible, therefore improving patient compliance. The technology described herein can be utilized with any therapeutic agent having an amine group.

### III. EXAMPLES

The following examples further illustrate the invention described herein and are in no way intended to limit the scope of the invention.

### Example 1

### Preparation of Polymer-DTB-Therapeutic Agent Conjugate

This reaction scheme is illustrated in part in FIG. 2A.

A. mPEG-DTB-Lysozyme

mPEG-methylDTB-nitrophenylcarbonates of various molecular weights (5-30 kDa) were prepared as described in Example 2A of U.S. Pat. No. 6,605,299, which is incorporated by reference herein. The structure of the mPEG-Me-NPC conjugate is shown in FIG. 2A, where R is CH₃ (methyl).

Lysozyme (at final concentration of 10 mg/mL) was allowed to react in borate buffer (0.1 M, pH 8.0) at 25° C. for 2-5 h with either mPEG-DTB-NPC or mPEG-NPC, using the feed molar ratio of 3.5 PEG/lysozyme (0.5 PEG/amino group). The conjugation reactions were quenched by the addition of 10-fold excess of glycine.

PEG-lysozyme conjugates were purified on a carboxymethyl HEMA-IEC Bio 1000 semi-preparative HPLC column (7.5×150 mm) purchased from Alltech Associates, Deerfield. IL. First, the conjugation reaction was injected into the HPLC column in 10 mM sodium acetate buffer pH 6. The elution with this buffer was continued until all unreacted PEG was removed. Then 0.2 M NaCl in 10 mM sodium acetate pH 6 was applied for 15 minutes in order to elute the PEGylated-lysozyme. Finally, the native lysozyme was eluted by increasing the salt concentration to 0.5 M NaCl over 20 min. Fractions (1 mL) were collected and assayed for protein and PEG contents. Thus aliquots (25 μL) of each fraction were reacted with BCA protein assay reagent (200 μL, Pierce Chemical Company, Rockford, Ill.) in microtiter plate wells at 37° C. for 30 min, and the absorbance was read at 562 nm. Similarly, for PEG determination, 25 μL aliquots were reacted with 0.1% polymethacrylic acid solution in 1 N HCl (200 μL) [S. Zalipsky & S. Menon-Rudolph (1997) Chapter 21, in Poly(ethylene Glycol): Chemistry and Biological Applications (J. M. Harris & S. Zalipsky, eds.), ACS Symposium Series 680, Washington, D.C., pp. 318-341], in microtiter plate wells, followed by absorbance reading at 400 nm. Fractions containing both protein and PEG were pooled. For the isolation of the PEG-lysozyme containing only one PEG moiety, the same cation exchange chromatography protocol was used, and the collected fractions were analyzed by the HPLC reversed-phase assay. Fractions containing the single peak of 1:1 PEG per lysozyme conjugate species were pooled.

B. mPEG-DTB-EPO

mPEG-MeDTB-nitrophenylcarbonates of various molecular weights (5-30 kDa) were prepared as described in Example 2A of U.S. Pat. No. 6,605,299, which is incorporated by reference herein.

Stock solutions of 16 mM mPEG-DTB-NPC (199.6 mg/mL) and mPEG-NPC (195.3 mg/mL) in acetonitrile were prepared.

Recombinant, human erythropoietin (EPO, EPREX®) was obtained preformulated at a protein concentration of 2.77 mg/mL in 20 mM Na citrate, 100 mM NaCl buffer pH 6.9.

mPEG-DTB-NPC was mixed with Epo at a 6:1 molar ratio in 50 mM MOPS, pH 7.8 for 4 hours at room temperature (approximately 25° C.). The reaction was further incubated at 4° C. overnight and then quenched by dialyzing in 10 mM Tris buffer, pH 7.5.

Prior to purification, the conjugates were dialyzed in 20 mM Tris pH 7.5 buffer and filtered through 0.2 μm Acrodisc® HT Tuffryn low protein binding syringe filter. The purification was done on a 1 mL Q XL anion exchanger column obtained from Amersham Biosciences Corp. (Piscataway, N.J.), using a step gradient elution profile from mobile phase A containing 20 mM Tris pH 7.5 buffer, to mobile phase B containing 500 mM NaCl in 20 mM Tris pH 7.5 buffer. The gradient was: 100% A for 8 minutes, 18% B for 25 minutes, then 70% B for 10 minutes. Elution fractions were collected in polypropylene tubes at 1 mL per fraction. The fractions eluting at 18% of mobile phase B (90 mM NaCl) were identified as the purified conjugates fractions (10 fractions), pooled in one tube, and stored at 2-8° C.

The purified mPEG-DTB-EPO conjugates were dialyzed in 20 mM sodium citrate, 100 mM NaCl buffer pH 6.9 (4 exchanges of 4 L buffer), using a Spectra/Por 6000-8000 MW cutoff dialysis tubing. A 10 mL Amicon concentrator with a YM10 membrane were used to bring down each sample volume from 10 to approximately 4.5 mL, under 45-50 psi nitrogen pressure.

### Example 2

### Decomposition of PEG-DTB-Protein Conjugates in Cysteine and BSA Solutions

Conjugates of PEG-DTB-lysozyme were prepared as described in Example 1A. The conjugates (100 μg/mL=0.066 mM) were incubated in 0.6 mM cysteine or with 4% BSA at room temperature (22-24° C.), in 10 mM phosphate buffer pH 7.4 containing 2 mM EDTA. Aliquots were taken at various time points, reactions were stopped with 20 mM iodoacetamide, and stored at 2-8° C. until analysis.

For the conjugates incubated with cysteine, analysis of the aliquots was as done follows. The samples were diluted 1/10 in 10 mM NaPO₄ pH 7.4 and analyzed on a carboxymethyl (CM) cation exchanger column.

For the conjugates incubated with BSA, analysis of the samples was done by diluting the samples 1/10 in 10 mM PO₄ pH 7.4, passing through Q spin columns (Vivascience) in order to trap the albumin and any of its related products, and then analyzing on the same CM column.

HPLC was performed with the following conditions: Column: TOSOH TSK CM-5PW 10 micron (7.5 mm×7.5 cm); Mobile phase: (A) 10 mM NaPO₄ pH 7.4 and (B) 500 mM NaCl in 10 mM NaPO₄ pH 7.4; Gradient: 5 min 100% A, 20 min 0% B to 100% B; Flow rate: 1 mL/min; Fluorescence detector: λex 295 nm, λem 360 nm (slit 30 nm); and injection volume, 100 μL.

The results are shown in FIGS. 3A-3D, FIGS. 4A-4D, and FIGS. 5A-5D.

### Example 3

### Identification of Albumin-Lysozyme Product Following Cleavage of mPEG-DTB-lysozyme with Albumin

A. Analysis by HPLC

mPEG5k-DTB-lysozyme 1-1 conjugate (100 μg/mL), prepared as described in Example 1, was incubated with 4% bovine serum albumin in 10 mM NaPO₄, 2 mM EDTA buffer, pH 7.4, for 2 days, at room temperature (22-24° C.). The reaction was then injected on a carboxymethyl (CM) cation exchanger column, and 0.5 mL fractions were collected and analyzed. The ion exchange separation conditions were: Column: HEMA CM 6.6 mL; Mobile Phase: A) 10 mM NaPO₄ pH 7.4, B) 500 mM NaCl in 10 mM NaPO₄ pH 7.4; Gradient: 10 min 100% A, 40 min 0% B to 100% B, then 1 min at 100% B; Flow rate: 1 mL/min; UV detector: 215 nm and 280 nm; injection volume, 3.3 mL. The HPLC trace is shown in FIG. 6.

B. Analysis by SDS-PAGE and by MALDI-TOFMS

Polyacrylamide gel electrophoresis under denaturing conditions was performed for conjugates characterization. Pre-cast NuPAGE® Bis-Tris gels (4-15%), NuPAGE® MES running buffer, molecular weight protein standards (Mark12™), and Colloidal Coomassie® G-250 staining kit, were all obtained from Invitrogen, Carlsbad, Calif. In a typical electrophoresis, 1 to 3 μg of protein containing sample were loaded per well on the gel, then electrophoresed at constant voltage of 200 mV, and stained for protein according to the manufacturer instructions. For PEG detection, a duplicate gel was stained with iodine according to Kurfürst. M., Anal. Biochem., 200(2):244-248 (1992). Fractions collected from the CM column separation were analyzed by SDS-PAGE gel as shown in FIG. 7.

The fractions collected from the CM column separation also incubated with 50 mM β-mercaptoethanol and then analyzed by SDS-PAGE again. The gel is shown in FIG. 8. Fractions E2 and E3 proved to be Albumin-Lysozyme adduct; fraction F1 was remaining mPEG-DTB-lysozyme (1:1) conjugate; Fraction G2 contained Iysozyme; fraction G4 corresponded to disulfide (DTB)-linked lysozyme dimmer. Similarly presence of albumin-lysozyme was identified from albumin-mediated reactions of other molecular weight PEG-DTB-lysozyme conjugates.

The purified albumin-lysozyme adduct (fraction E2 in FIG. 6) was analyzed by MALDI-TOFMS, and the molecular ion of the main albumin-lysozyme adduct of 81 kDa was present as shown in FIG. 9.

### Example 4

### Characterization of Decomposition of Polymer-DTB-Protein Conjugates in Plasma and Albumin Solutions

mPEG-DTB-lysozyme and mPEG-DTB-erythropoietin conjugates derived from mPEG of molecular weight 5, 12 and 30 kDa were prepared as described above. The conjugates were labeled with Alexa Fluor™ 488 and free dye was removed. Labeled conjugates (0.05-0.1 mg/mL) were incubated with 75% rat plasma or with 3.55% bovine serum albumin (BSA) in the presence of phosphate buffered saline, pH 7.4. Samples withdrawn for analysis at a specified time point were treated with 50 mM iodoacetamide to terminate the cleavage of the disulfide and then placed on ice. Collected samples were analyzed by SDS PAGE and the Alexa Fluor™ 488 fluorophore image was quantitated using a fluorescence imager. The results are shown in FIGS. 10-13.

### Example 5

### Characterization of Polymer-DTB-Erythropoietin Conjugate

A. Cleavage of Conjugate in Cysteine and in HSA

mPEG-DTB-Epo (prepared as described above), mPEG-Epo, or Epo (0.2 mg/mL) was incubated with 0.05% human serum albumin (HSA) in 100 mM HEPES, 2 mM EDTA, pH 7.5 buffer for 21 hours at 37° C. To ensure visualization of the reaction products by SDS-PAGE, the concentration of HSA was significantly lower than physiological conditions and small molecule thiols were not included in the reaction, to prevent subsequent cleavage of any formed albumin-Epo. The SDS-PAGE gel stained with SYPRO™ red protein stain is shown in FIG. 14A and an immunoblot probed with anti-HSA is shown in FIG. 14B.

B. Cleavage of Fluorescent Conjugates in Rat Plasma and in BSA

Fluorescently labeled mPEG-DTB-protein conjugates were also observed in the presence of rat plasma or bovine serum albumin over a timecourse at 37° C. mPEG-DTB-Epo conjugates were labeled and purified using the Alexa Fluor™ 488 labeling kit from Molecular Probes (Eugene, Oreg.), essentially according to kit instructions. Plasma from Sprague Dawley rats was collected with EDTA as the anticoagulant and stored in aliquots at −20° C. Bovine serum albumin from Proliant (Ankeny, IA) was resuspended in 50 mM NaPO4/2 mM EDTA, pH 7.4. Reactions contained 75% plasma or 3.5% BSA, 0.05-0.1 mg/mL labeled conjugate protein (1.6-3.3 μM for Epo; 3.5-7 μM for lysozyme) and phosphate buffered saline, pH 7.4 in tubes with o-ring caps. Samples were taken from each reaction mixture and stopped with 50 mM iodoacetamide (150 mM stock concentration in 50 mM NaPO₄/2 mM EDTA), and placed on ice, protected from light. For time zero samples, plasma or BSA was quenched with iodoacetamide prior to addition of fluorescent mPEG-DTB-protein.

Collected samples were separated on NuPAGE™ 4-12% gels (Invitrogen, Carlsbad, Calif.) with MOPS or MES running buffer in presence of excess NuPAGE™ loading buffer. Prestained molecular weight markers were from Invitrogen (Carlsbad, Calif.). Imaging and quantitation was done using the Typhoon™ 9400 and ImageQuant™ (Amersham Biosciences) at λex=488 nm, λem=520 nm band pass 40. Following Alexa Fluor™ 488 quantitation, total protein signal was imaged (at λex=488 nm, λem=610 nm band pass 30) after staining with SYPRO™ red (Amersham Biosciences). The percent of each species compared to the total Alexa Fluor™ 488 labeled material was determined for each lane. Results are shown in FIGS. 15-17.

### Example 6

### Characterization of Polymer-DTB-Lysine-NBD Conjugate in the Presence of Albumin

mPEG_{30K}-DTB-Lysine-NBD prepared similarly to Example 1 above using 2 mM mPEG_{30K}-DTB-nitrophenylcarbonate and 5-fold molar excess H-Lys-(ε-NBD)-NH₂ (custom synthesized by Anaspec, San Jose, Calif.) in the presence of 60 mM hydroxysuccinimide, 60 mM HEPES, pH 7.5. Non-cleavable mPEG_{30K}-Lysine-NBD was prepared using PEG_{30K}-succinimidyl carbonate. In both preparations, free H-Lys-(ε-NBD)-NH₂ was removed by Sephadex G-25 in PBS, pH 7.4. Cleavage reactions with BSA and analysis were essentially as described in Example 5B using 3.3% BSA in an equimolar ratio to the PEG reagent. Higher ratios of PEG reagents led to high background from the PEG reagent. When lower ratios of PEG reagent were used, the reagent was completely consumed in the reaction with time, but detection was low. An equimolar ratio allowed optimal visualization for quantifying the NBD (7-nitrobenz-2-oxa-1,3-diazole) fluorophore by SDS-PAGE and fluorescence imaging at λex=488 nm, λem=555 nm band pass 20. The results are shown in FIGS. 18-20 with FIGS. 18 and 19A-19C showing a 3-8% Tris-Acetate gel used according to the manufacturer (Invitrogen, Carlsbad, Calif.). Gels were stained with Simply Blue™ (Invitrogen) for protein visualization and with iodine for PEG visualization.

### Example 7

### Cleavage of mPEG₅ₖ-DTB-Lysozyme (1-1 Conjugate) in Cysteine and BSA. Analysis by Micrococcus luteus Turbidity Assay

A conjugate of mPEG₅ₖ-DTB-lysozyme was purified and prepared as a stock solution of 2.56 mg/mL. The solution contained 96% of pure 1-1 mPEG-protein conjugate, 1.6% of 2-1 conjugate, and approximately 2% of unconjugated lysozyme. A Micrococcus luteus turbidity assay was used to measure the amount of active lysozyme regenerated after cleavage of the conjugate.

mPEG₅ₖ-DTB-lysozyme (50 μg/mL in protein concentration) was incubated with 0.6 mM cysteine and with 4% BSA (containing approximately 0.45 mM free thiol, assuming that 75% of the albumin was in free SH form), at 37° C., in 10 mM NaPO₄/140 mM NaCl/2 mM EDTA pH 7.4 buffer. At various time points, aliquots from the incubation vials were added to iodoacetamide to a final concentration of 20 mM, in order to stop the cleavage reaction. Samples were stored at 2-8° C. prior to analysis.

Micrococcus luteus stock solution was prepared at 0.3 mg/mL in 100 mM KPO₄ pH 7. Lysozyme standards solutions were prepared at 1, 2, 4, 6, 8, and 10 μg/mL in PBS and a lysozyme standard curve was constructed (not shown). The samples from the cleavage reactions were diluted 1/10 in PBS. For the assay, 50 μL of standard, sample, or control were added per well to 96-well microtiter plates. To each well, 200 μL of Micrococcus luteus were added, and without delay, plates were read at 450 nm at 25° C. in a plate reader of a period of 10 min, in 30 second reading intervals.

The slopes (ΔA/min) were calculated for the first 5 minutes of the reading, and the corresponding lysozyme concentrations were extrapolated from the lysozyme standard curve. The results are shown graphically in FIG. 21 for the conjugates cleaved in cysteine (squares), BSA (circles), or PBS (triangles).

### Example 8

### In Vivo Administration of Polymer-DTB-Therapeutic Agent Conjugate

A. Preparation of ¹²⁵I PEG-Lysozyme

Lysozyme (66 mg in 100 mg/ml in 0.1 M sodium phosphate buffer pH 7.3) was mixed with 605 μCi of Na¹²⁵I (ICN Biomedicals, Irvine, Calif.), in Iodo-Gen® coated tube (Pierce Chemical Company, Rockford, Ill.), and allowed to react for 1 hour at room temperature with 20 min intervals mixing. The iodination reaction was stopped by removing the free ¹²⁵I on a Sephadex G-25F gel filtration column (17 mL), and collecting the ¹²⁵I-lysozyme, which was then reacted with either mPEG-DTB-NPC and mPEG-NPC, and purified by cation exchange chromatography as described above.

B. Pharmacokinetic Experiments

Male Sprague-Dawley rats (250-330 g each, 3 animals per formulation per experiment) were dosed either by intravenous (via a lateral tail vein) or by subcutaneous (dorsally above the right rear leg) with ¹²⁵I labeled lysozyme or its PEG conjugates (0.35 mL, 0.4 mg protein/mL, 4.6×10⁶ cpm/mL). Blood samples (0.4 mL) were collected via the retro-orbital sinus. All injections blood collections were performed while the animals were under inhaled anesthesia (isoflurane/O₂). Samples were collected on heparin into polypropylene tubes and stored on ice for no longer than one hour before being pipetted in triplicate (0.100 mL) into fresh polypropylene tubes. Blood samples were collected at the following times after dosing (no single rat had blood collected at all of the following times): 30 sec, 15 min, 30 min and 1, 2, 3, 4, 6, 8, 24, 48, 72, 96, 120 and 168 hours post-dose. Note that the last 4 time points were added for the longer subcutaneous experiments. The samples were then counted for ¹²⁵I in a Packard™ 5000 gamma counter. The cpm counts were converted to concentration according to the specific activity of the samples.

The results are shown in FIG. 22.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions and sub-combinations as are within their true spirit and scope.

It is claimed:

1. A method for delivering a therapeutic agent in the form of a conjugate with albumin, comprising
administering to a subject a compound of the form polymer-disulfide-therapeutic agent, wherein said therapeutic agent comprises at least one amine moiety;
whereby said administering achieves formation of a conjugate comprised of the subject's endogenous albumin and the therapeutic agent.

2. The method according to claim 1, wherein said polymer-disulfide-therapeutic agent compound has the structure: 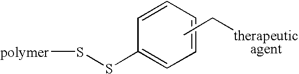
where orientation of CH₂-therapeutic agent is selected from the ortho position and the para position.

3. The method according to claim 1, wherein said amine-containing therapeutic agent is selected from a protein and a drug.

4. The method according to claim 2, wherein said amine-containing therapeutic agent is selected from a protein and a drug.

5. The method according to claim 4, wherein said therapeutic agent has a molecular weight of less than about 45 kDa.

6. The method according to claim 4, wherein said polymer is polyethylene glycol.

7. A prodrug for treatment of a subject, the prodrug comprised of the subject's endogenous albumin and a therapeutic agent comprising at least one amine moiety, said albumin and said therapeutic agent joined by a disulfide, said prodrug being obtainable by administering to the subject a polymer-disulfide-therapeutic agent conjugate, wherein at least about 35% of the therapeutic agent administered in the form of said polymer-disulfide-therapeutic agent conjugate is converted to said prodrug.

8. The prodrug of claim 7, wherein said therapeutic agent has a molecular weight of less than 45 kDa.

9. The prodrug of claim 7, wherein said polymer is polyethylene glycol.

10. The prodrug of claim 7, wherein said therapeutic agent is a drug.

11. The prodrug of claim 7, wherein said therapeutic agent is a polypeptide.

12. The prodrug of claim 7 having the form 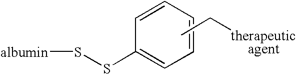
where orientation of CH₂-therapeutic agent is selected from the ortho position and the para position.

13. A method for extending the blood circulation lifetime of a therapeutic agent, comprising
administering to a subject a compound of the form a polymer-DTB-therapeutic agent conjugate, wherein said therapeutic agent comprises at least one amine moiety;
whereby said administering achieves formation of a prodrug conjugate comprised of endogenous albumin and said therapeutic agent, and said prodrug conjugate has a blood circulation lifetime greater than the blood circulation lifetime of the therapeutic agent when administered in free form.

14. The method according to claim 13, wherein said polymer is polyethylene glycol.

15. The method of claim 13, wherein said therapeutic agent has a molecular weight of less than 45 kDa.

16. The method of claim 13, wherein said therapeutic agent is a drug.

17. The method of claim 13, wherein said therapeutic agent is a polypeptide.

18. The method of claim 13 having the form 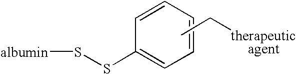
where orientation of CH₂-therapeutic agent is selected from the ortho position and the para position.
